(19) United States
(12) Patent Application Publication
(10) Pub. No.: US 2026/0085119 A1
(43) Pub. Date: Mar. 26, 2026

(54) METHODS FOR TREATING CANCER USING ANTI-CTLA4 ANTIBODIES

(71) Applicant: ADAGENE PTE. LTD., Singapore (SG)

(72) Inventors: Peter Peizhi LUO, Morgan Hill, CA (US); Songmao ZHENG, Morgan Hill, CA (US); Jiping ZHA, Morgan Hill, CA (US); Guizhong LIU, Morgan Hill, CA (US); Xiaohong SHE, Morgan Hill, CA (US)

(73) Assignee: ADAGENE PTE. LTD., Singapore (SG)

(21) Appl. No.: 19/106,860

(22) PCT Filed: Aug. 30, 2023
(86) PCT No.: PCT/US2023/073182

§ 371 Date: Feb. 26, 2025

Related U.S. Application Data
(60) Provisional application No. 63/402,247, filed on Aug. 30, 2022.

(51) Int. Cl. C07K 16/28; A61K 39/00; A61P 35/00

(52) U.S. Cl. CPC C07K 16/2818; A61P 35/00; A61K 2039/507; A61K 2039/545; C07K 2317/24; C07K 2317/73; C07K 2317/76; C07K 2317/92; C07K 2317/94

(57) ABSTRACT

The present application provides compositions and methods for treating cancers, including cancers that are resistant or refractory to an inhibitor of PD-1 or PD-L 1, using an anti-CTLA4 antibody, including combination therapies.

### CROSS-REFERENCE TO RELATED APPLICATIONS

This application claims priority to U.S. Provisional Patent Application No. 63/402,247 filed Aug. 30, 2022, the disclosure of which is hereby incorporated by reference in its entirety for all purposes.

### REFERENCE TO AN ELECTRONIC SEQUENCE LISTING

The contents of the electronic sequence listing (695402002540SEQLIST.xml; Size: 187,921 bytes; and Date of Creation: Aug. 24, 2023) is herein incorporated by reference in its entirety.

### FIELD OF THE INVENTION

The present application is in the field of cancer therapeutics, and relates to compositions and methods for treating cancers using antibodies that bind to human CTLA4.

### BACKGROUND

CTLA4 is a member of the immunoglobulin (Ig) superfamily of proteins that acts to downregulate T-cell activation and maintain immunogenic homeostasis. It has been shown that in vivo antibody-mediated blockade of CTLA4 enhanced anti-cancer immune responses in a syngeneic murine prostate cancer model (Kwon et al. (1997) Proc Natl Acad Sci USA, 94(15):8099-103). In addition, blockade of CTLA4 function was shown to enhance anti-tumor T cell responses at various stages of tumor growth in tumor-bearing mice (Yang et al. (1997) Cancer Res 57(18):4036-41; Hurwitz et al. (1998) Proc Natl Acad Sci USA 95 (17):10067-7). However, the development of antibody-based therapeutics suitable for human use remains difficult, as translation from pre-clinical animal models to human safety is often poor. Accordingly, a need exists for anti-CTLA4 antibodies that are cross-reactive among different species, such as humans and experimental animals (e.g., mouse, monkey, rat, etc.), to concurrently enable animal model studies and provide suitable human therapeutic candidates. In addition, a need exists for the development of safer anti-CTLA4 antibodies that are only active in certain contexts, such as in the protease-rich tumor microenvironment.

### BRIEF SUMMARY

The present application provides methods for treating cancer with an anti-CTLA4 antibody, wherein the antibody specifically binds to an epitope comprising amino acid residues Y105 and L106 of human CTLA4 but does not comprise residue 1108. The present application also provides methods of treating cancer with an anti-CTLA4 antibody of the disclosure in combination with one or more therapeutic agents, and in particular, an anti-PD-1 antibody.

In some embodiments according to any one of the methods described above, the antibody is a human antibody. In some embodiments, the antibody comprises an IgG1, IgG2, IgG3, or IgG4 Fc region (such as human IgG1, IgG2, IgG3, or IgG4 Fc region). In some embodiments, the antibody comprising a human IgG1 or a variant that has enhanced ADCC activity. In some embodiments, the antibody comprises a human IgG1 with reduced fucosylation (or non-fucosylated).

In some embodiments, the anti-CTLA4 antibody comprising a heavy chain variable region and a light chain variable region, wherein the heavy chain variable region comprises an HVR-H1, an HVR-H2, and an HVR-H3, and the light chain variable region comprises an HVR-L1, an HVR-L2, and an HVR-L3, wherein the HVR-H1 comprises an amino acid sequence according to a formula YSISSGYHWSWI (SEQ ID NO: 23), the HVR-H2 comprises an amino acid sequence according to a formula LARIDWDDDKYYSTSLKSRL (SEQ ID NO: 35), the HVR-H3 comprises an amino acid sequence according to a formula ARSYVYFDY (SEQ ID NO: 45), the HVR-L1 comprises an amino acid sequence according to a formula RASQSVRGRFLA (SEQ ID NO: 58), the HVR-L2 comprises an amino acid sequence according to a formula DASNRATGI (SEQ ID NO: 66), and the HVR-L3 comprises an amino acid sequence according to a formula YCQQSSSWPPT (SEQ ID NO: 75).

In some embodiments, the anti-CTLA4 antibody comprises a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100. In some embodiments, the anti-CTLA4 antibody comprises a heavy chain variable region comprising the amino acid sequence of SEQ ID NO: 87 and light chain variable region comprising the amino acid sequence of SEQ ID NO: 100.

In some embodiments, the anti-CTLA4 antibody comprises a heavy chain region comprising the amino acid sequence of EVQLVESGGGLVQPGGSLRLSCAASGYSISSGYHWSWIRQAPGKGLEWLARIDWDDDKYYSTSL KSRLTISRDNSKNTLYLQLNSLRAEDTAVYYCARSYVYFDYWGQGTLVTVSSASTKGPSVFPLA PSSKSTSGGTAALGCLVKDYFPEPVTVSWNSGALTSGVHTFPAVLQSSGLYSLSSVVTVPSSSL GTQTYICNVNHKPSNTKVDKKVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTQKSLSLSPGK (SEQ ID NO:126) or a variant thereof having at least about 90% (e.g., at least about 92%, 95%, 98%, 99% or more) sequence identity to the amino acid sequence of SEQ ID NO: 126, and a light chain region comprising the amino acid sequence of DIQLTQSPSSLSASVGDRVTITCRASQSVRGRFLAWYQQKPGKAPKLLIYDASNRATGIPSRFS GSGSGTDFTLTISSLQPEDFATYYCQQSSSWPPTFGQGTKVEIKRTVAAPSVFIFPPSDEQLKS GTASVVCLLNNFYPREAKVQWKVDNALQSGNSQESVTEQDSKDSTYSLSSTLTLSKADYEKHKV YACEVTHQGLSSPVTKSFNRGEC (SEQ ID NO:127) or a variant thereof having at least about 90% (e.g., at least about 92%, 95%, 98%, 99% or more) sequence identity to the amino acid sequence of SEQ ID NO:127. In some embodiments, the anti-CTLA4 antibody is TY21580, which comprises a heavy chain region comprising the amino acid sequence of SEQ ID NO: 126 and light chain region comprising the amino acid sequence of SEQ ID NO: 127.

In one aspect, the disclosure provides a method of treating a cancer in a subject, comprising administering to the subject an effective amount of an anti-CTLA4 described above (e.g., TY21580) as a monotherapy at a dose of from about 3 mg/kg to about 15 mg/kg. In some embodiments, the anti-CTLA4 (e.g., TY21580) is administered at a dose of about 3 mg/kg. In some embodiments, the anti-CTLA4 (e.g., TY21580) is administered at a dose of about 5 mg/kg. In some embodiments, the anti-CTLA4 (e.g., TY21580) is administered at a dose of about 6 mg/kg. In some embodiments, the anti-CTLA4 (e.g., TY21580) is administered at a dose of about 8 mg/kg. In some embodiments, the anti-CTLA4 (e.g., TY21580) is administered at a dose of about 10 mg/kg. In some embodiments, the anti-CTLA4 (e.g., TY21580) is administered at a dose of about 15 mg/kg.

In another aspect, the disclosure provides a method of treating a cancer in a subject, comprising administering to the subject: (a) an effective amount of an anti-CTLA4 described above (e.g., TY21580), and (b) an effective amount of an anti-PD-1 antibody. In one embodiment, wherein the antibody comprises a heavy chain variable region comprising the amino acid sequence of SEQ ID NO: 87 and light chain variable region comprising the amino acid sequence of SEQ ID NO: 100, the antibody can be administered to the subject at dose of from about 1 mg/kg to about 10 mg/kg or from about 2 mg/kg to about 5 mg/kg. In some such embodiments, the anti-CTLA4 antibody can be administered to the subject at a dose of about 3 mg/kg. In some embodiments, the anti-CTLA4 antibody can be administered to the subject once every three weeks. In other embodiments, the anti-CTLA4 antibody can be administered to the subject once every six weeks. In particular embodiments, the antibody TY21580 can be administered to a patient at a dose of about 3 mg/kg once every three weeks or once every six weeks.

In some embodiments according to any one of the methods described above, the cancer is resistant or refractory to a prior therapy, wherein the prior therapy is an inhibitor of CTLA4, PD-1, or a PD-1 ligand. In some embodiments, the subject is resistant to or has relapsed from a prior therapy, wherein the prior therapy is an inhibitor of CTLA4, PD-1, or a PD-1 ligand. In some embodiments, the prior therapy is an inhibitor of CTLA4, such as ipilimumab. In some embodiments, the prior therapy is an inhibitor of PD-1, such as an anti-PD-1 antibody. In some embodiments, the prior therapy is an inhibitor of a PD-1 ligand (e.g., PD-L1), for example an anti-PD-L1 antibody.

Another aspect of the present application provides a method of treating a cancer in a subject, comprising administering to the subject an effective amount of an anti-CTLA4 antibody described above in combination with an anti-PD-1 antibody, wherein the numbering of the amino acid residues is according to SEQ ID NO: 108, and wherein the cancer is resistant or refractory to a prior therapy, wherein the prior therapy is an inhibitor of CTLA4, PD-1, or a PD-1 ligand. In some such embodiments the anti-CTLA4 antibody is TY21580.

In some embodiments according to any one of the methods described above, the cancer is liver cancer, a cancer of the digestive system (e.g., colon cancer, colorectal cancer), lung cancer, bone cancer, heart cancer, brain cancer, kidney cancer, bladder cancer, a hematological cancer (e.g., leukemia), skin cancer, breast cancer, thyroid cancer, pancreatic cancer, a head and/or neck cancer, an eye-related cancer, a male reproductive system cancer (e.g., prostate cancer, testicular cancer), or a female reproductive system cancer (e.g., uterine cancer, cervical cancer). In some embodiments, the cancer is a solid cancer. In some embodiments, the cancer is urothelial carcinoma. In some embodiments, the cancer is renal cell carcinoma. In some embodiments, the cancer is pancreatic cancer. In some embodiments, the cancer is advanced-stage cancer. In some embodiments, the cancer is metastatic cancer. In some embodiments, the cancer is Kaposi's sarcoma. In some embodiments, head and neck squamous cell carcinoma (HNSCC).

In some embodiments, the anti-CTLA4 antibody is administered intravenously. In some embodiments, the anti-CTLA4 antibody is administered subcutaneously. In some embodiments, the anti-CTLA4 antibody is administered intravenously or subcutaneously once every three weeks. In some embodiments, the anti-CTLA4 antibody is administered intravenously or subcutaneously once every six weeks. In some embodiments, the subject receives at least 4 cycles of treatment with the anti-CTLA4 antibody. In some embodiments, the subject further receives a maintenance treatment comprising administering to the subject an effective amount of the anti-CTLA4 antibody about once every four weeks to about once every twelve weeks (e.g., once every 4, 6, 8, 10, or 12 weeks).

In some embodiments according to any one of the methods described above, the subject is human.

It is to be understood that one, some, or all of the properties of the various embodiments described above and herein may be combined to form other embodiments of the present application. These and other aspects of the present application will become apparent to one of skill in the art. These and other embodiments of the present application are further described by the detailed description that follows.

### BRIEF DESCRIPTION OF FIGURES

FIG. 1 shows TY21580 observed cycle 1 and steady-state PK at 3 mg/kg (Q3W or Q6W dosing) vs. Ipilimumab steady-state PK at 1 mg/kg (Q6W dosing)

### DETAILED DESCRIPTION

### I. Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular. Generally, nomenclatures used in connection with, and techniques of, antibody engineering, immunotherapy, cell and tissue culture, molecular biology, immunology, microbiology, genetics and protein and nucleic acid chemistry described herein are those well-known and commonly used in the art.

The term “antibody” is used herein in the broadest sense and specifically covers monoclonal antibodies (including full length monoclonal antibodies), polyclonal antibodies, multispecific antibodies (e.g., bispecific antibodies, trispecific antibodies), and antibody fragments (e.g., Fab, Fab′, Fab′-SH, F(ab′)₂, Fv and/or a single-chain variable fragment or scFv) so long as they exhibit the desired biological activity.

In some embodiments, the term “antibody” refers to an antigen-binding protein (i.e., immunoglobulin) having a basic four-polypeptide chain structure consisting of two identical heavy (H) chains and two identical light (L) chains. Each L chain is linked to an H chain by one covalent disulfide bond, while the two H chains are linked to each other by one or more disulfide bonds depending on the H chain isotype. Each heavy chain has, at the N-terminus, a variable region (abbreviated herein as V_{H}) followed by a constant region. The heavy chain constant region is comprised of three domains, C_{H1}, C_{H2} and C_{H3}. Each light chain has, at the N-terminus, a variable region (abbreviated herein as V_{H}) followed by a constant region at its other end. The light chain constant region is comprised of one domain, C_{L}. The V_{L} is aligned with the V_{H} and the C_{L} is aligned with the first constant domain of the heavy chain (CH1). The pairing of a V_{H} and V_{L} together forms a single antigen-binding site. An IgM antibody consists of 5 of the basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain.

The V_{H} and V_{L}regions can be further subdivided into regions of hypervariability, termed hyper-variable regions (HVR) based on structural and sequence analysis. HVRs are interspersed with regions that are more conserved, termed framework regions (FW) (see e.g., Chen et al. (1999) J. Mol. Biol. (1999) 293, 865-881). Each V_{H} and V_{L} is composed of three HVRs and four FWs, arranged from amino-terminus to carboxy-terminus in the following order: FW-1_HVR-1_FW-2_HVR-2_FW-3_HVR-3_FW4. Throughout the present application, the three HVRs of the heavy chain are referred to as HVR-H1, HVR-H2, and HVR-H3. Similarly, the three HVRs of the light chain are referred to as HVR-L1, HVR-L2, and HVR-L3.

As used herein, the term “CDR” or “complementarity determining region” is intended to mean the non-contiguous antigen combining sites found within the variable region of both heavy and light chain polypeptides. These particular regions have been described by Kabat et al., J. Biol. Chem. 252:6609-6616 (1977); Kabat et al., U.S. Dept. of Health and Human Services, “Sequences of proteins of immunological interest” (1991); Chothia et al., J. Mol. Biol. 196:901-917 (1987); Al-Lazikani B. et al., J. Mol. Biol., 273: 927-948 (1997); MacCallum et al., J. Mol. Biol. 262:732-745 (1996); Abhinandan and Martin, Mol. Immunol., 45: 3832-3839 (2008); Lefranc M. P. et al., Dev. Comp. Immunol., 27: 55-77 (2003); and Honegger and Pluckthun, J. Mol. Biol., 309:657-670 (2001), where the definitions include overlapping or subsets of amino acid residues when compared against each other. Nevertheless, application of either definition to refer to a CDR of an antibody or grafted antibodies or variants thereof is intended to be within the scope of the term as defined and used herein. CDR prediction algorithms and interfaces are known in the art, including, for example, Abhinandan and Martin, Mol. Immunol., 45: 3832-3839 (2008); Ehrenmann F. et al., Nucleic Acids Res., 38: D301-D307 (2010); and Adolf-Bryfogle J. et al., Nucleic Acids Res., 43: D432-D438 (2015). The contents of the references cited in this paragraph are incorporated herein by reference in their entireties for use in the present invention and for possible inclusion in one or more claims herein.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. Within light and heavy chains, the variable and constant regions are joined by a “J” region of about 12 or more amino acids, with the heavy chain also including a “D” region of about 10 or more amino acids (see e.g., Fundamental Immunology Ch. 7 (Paul, W., ed., 2^{nd} ed. Raven Press, N.Y). (1989)).

The L chain from any vertebrate species can be assigned to one of two clearly distinct types, called kappa and lambda, based on the amino acid sequences of their constant domains. Depending on the amino acid sequence of the constant domain of their heavy chains (CH), antibodies can be assigned to different classes or isotypes. There are five classes of antibodies: IgA, IgD, IgE, IgG, and IgM, having heavy chains designated α (alpha), δ (delta), F (epsilon), γ (gamma), and μ (mu), respectively. The IgG class of antibody can be further classified into four subclasses IgG1, IgG2, IgG3, and IgG4 by the gamma heavy chains, Y1-Y4, respectively.

The term “CTLA4” is used in the present application, and includes the human CTLA4 (e.g., UniProt accession number P16410), as well as variants, isoforms, and species homologs thereof (e.g., mouse CTLA4 (UniProt accession number P09793), rat CTLA4 (UniProt accession number Q9Z1A7), dog CTLA4 (UniProt accession number Q9XSI1), cynomolgus monkey CTLA4 (UniProt accession number G7PL88), etc.). Accordingly, an anti-CTLA4 antibody, as defined and disclosed herein, may also bind CTLA4 from species other than human. In other cases, an anti-CTLA4 antibody may be completely specific for the human CTLA4 and may not exhibit species or other types of cross-reactivity.

The term “CTLA4 antibody” refers to an antibody, as defined herein, capable of binding to human CTLA4.

The term “epitope” refers to a part of an antigen to which an antibody (or antigen-binding fragment thereof) binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope can include various numbers of amino acids in a unique spatial conformation. Methods of determining spatial conformation of epitopes include, for example, x-ray crystallography, 2-dimensional nuclear magnetic resonance, deuterium and hydrogen exchange in combination with mass spectrometry, or site-directed mutagenesis, or all methods used in combination with computational modeling of antigen and its complex structure with its binding antibody and its variants (see e.g., Epitope Mapping Protocols in Methods in Molecular Biology, Vol. 66, G. E. Morris, Ed. (1996)). Once a desired epitope of an antigen is determined, antibodies to that epitope can be generated, e.g., using the techniques described herein. The generation and characterization of antibodies may also elucidate information about desirable epitopes. From this information, it is then possible to competitively screen antibodies for binding to the same epitope. An approach to achieve this is to conduct cross-competition studies to find antibodies that competitively bind with one another, i.e., the antibodies compete for binding to the antigen. A high throughput process for “binning” antibodies based upon their cross-competition is described in PCT Publication No. WO 03/48731.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see e.g., Flatman et al., J. Chromatogr. B 848:79-87 (2007).

As used herein, “sequence identity” between two polypeptide sequences indicates the percentage of amino acids that are identical between the sequences. The amino acid sequence identity of polypeptides can be determined conventionally using known computer programs such as Bestfit, FASTA, or BLAST (see e.g., Pearson, Methods Enzymol. 183:63-98 (1990); Pearson, Methods Mol. Biol. 132:185-219 (2000); Altschul et al., J. Mol. Biol. 215:403-410 (1990); Altschul et al., Nucleic Acids Res. 25:3389-3402 (1997)). When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed. This aforementioned method in determining the percentage of identity between polypeptides is applicable to all proteins, fragments, or variants thereof disclosed herein.

As used herein, the term “binds”, “binds to”, “specifically binds” “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤1 μM, ≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “treat”, “treating”, or “treatment”, with reference to a certain disease condition in a mammal, refers causing a desirable or beneficial effect in the mammal having the disease condition. The desirable or beneficial effect may include reduced frequency or severity of one or more symptoms of the disease (i.e., tumor growth and/or metastasis, or other effect mediated by the numbers and/or activity of immune cells, and the like), or arrest or inhibition of further development of the disease, condition, or disorder. In the context of treating cancer in a mammal, the desirable or beneficial effect may include inhibition of further growth or spread of cancer cells, death of cancer cells, inhibition of reoccurrence of cancer, reduction of pain associated with the cancer, or improved survival of the mammal. The effect can be either subjective or objective. For example, if the mammal is human, the human may note improved vigor or vitality or decreased pain as subjective symptoms of improvement or response to therapy. Alternatively, the clinician may notice a decrease in tumor size or tumor burden based on physical exam, laboratory parameters, tumor markers or radiographic findings. Some laboratory signs that the clinician may observe for response to treatment include normalization of tests, such as white blood cell count, red blood cell count, platelet count, erythrocyte sedimentation rate, and various enzyme levels.

Additionally, the clinician may observe a decrease in a detectable tumor marker. Alternatively, other tests can be used to evaluate objective improvement, such as sonograms, nuclear magnetic resonance testing and positron emissions testing.

The term “prevent” or “preventing,” with reference to a certain disease condition in a mammal, refers to preventing or delaying the onset of the disease, or preventing the manifestation of clinical or subclinical symptoms thereof.

As used herein, a “subject”, “patient”, or “individual” may refer to a human or a non-human animal. A “non-human animal” may refer to any animal not classified as a human, such as domestic, farm, or zoo animals, sports, pet animals (such as dogs, horses, cats, cows, etc.), as well as animals used in research. Research animals may refer without limitation to nematodes, arthropods, vertebrates, mammals, frogs, rodents (e.g., mice or rats), fish (e.g., zebrafish or pufferfish), birds (e.g., chickens), dogs, cats, and non-human primates (e.g., rhesus monkeys, cynomolgus monkeys, chimpanzees, etc.). In some embodiments, the subject, patient, or individual is a human.

An “effective amount” refers to at least an amount effective, at dosages and for periods of time necessary, to achieve one or more desired or indicated effects, including a therapeutic or prophylactic result. An effective amount can be provided in one or more administrations. For purposes of the present application, an effective amount of antibody, drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition (e.g., an effective amount as administered as a monotherapy or combination therapy). Thus, an “effective amount” may be considered in the context of administering one or more therapeutic agents, and a single agent may be considered to be given in an effective amount if, in conjunction with one or more other agents, a desirable result may be or is achieved.

The terms “recurrence,” “relapse” or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

As used herein, “complete response” or “CR” refers to disappearance of all target lesions; “partial response” or “PR” refers to at least a 30% decrease in the sum of the longest diameters (SLD) of target lesions, taking as reference the baseline SLD; and “stable disease” or “SD” refers to neither sufficient shrinkage of target lesions to qualify for PR, nor sufficient increase to qualify for PD, taking as reference the smallest SLD since the treatment started.

As used herein, “progressive disease” or “PD” refers to at least a 20% increase in the SLD of target lesions, taking as reference the smallest SLD recorded since the treatment started or the presence of one or more new lesions.

As used herein, “progression free survival” (PFS) refers to the length of time during and after treatment during which the disease being treated (e.g., cancer) does not get worse. Progression-free survival may include the amount of time patients have experienced a complete response or a partial response, as well as the amount of time patients have experienced stable disease.

As used herein, “overall response rate” (ORR) refers to the sum of complete response (CR) rate and partial response (PR) rate.

As used herein, “overall survival” refers to the percentage of individuals in a group who are likely to be alive after a particular duration of time.

As used herein, a “baseline level” or “baseline value” refers to a level or a value of a subject before the subject begins a treatment, such as an anti-CTLA4 antibody treatment.

A “reference sample”, “reference cell”, “reference tissue”, “control sample”, “control cell”, or “control tissue”, as used herein, refers to a sample, cell, tissue, standard, or level that is used for comparison purposes. In one embodiment, a reference sample, reference cell, reference tissue, control sample, control cell, or control tissue is obtained from a healthy and/or non-diseased part of the body (e.g., tissue or cells) of the same subject or individual. For example, healthy and/or non-diseased cells or tissue adjacent to the diseased cells or tissue (e.g., cells or tissue adjacent to a tumor). In another embodiment, a reference sample is obtained from an untreated tissue and/or cell of the body of the same subject or individual. In yet another embodiment, a reference sample, reference cell, reference tissue, control sample, control cell, or control tissue is obtained from a healthy and/or non-diseased part of the body (e.g., tissues or cells) of an individual who is not the subject or individual. In even another embodiment, a reference sample, reference cell, reference tissue, control sample, control cell, or control tissue is obtained from an untreated tissue and/or cell of the body of an individual who is not the subject or individual.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and progression free survival); resulting in an objective response (including a complete response or a partial response); or improving signs or symptoms of cancer.

A patient who “does not have an effective response” to treatment refers to a patient who does not have any one of extending survival (including overall survival and progression free survival); resulting in an objective response (including a complete response or a partial response); or improving signs or symptoms of cancer.

The methods and techniques of the present application are generally performed according to methods well known in the art and as described in various general and more specific references that are cited and discussed throughout the present specification unless otherwise indicated. Such references include, e.g., Sambrook and Russell, Molecular Cloning, A Laboratory Approach, Cold Spring Harbor Press, Cold Spring Harbor, N.Y. (2001), Ausubel et al., Current Protocols in Molecular Biology, John Wiley & Sons, NY (2002), and Harlow and Lane Antibodies: A Laboratory Manual, Cold Spring Harbor Laboratory Press, Cold Spring Harbor, N.Y. (1990). Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

As used herein, the twenty conventional amino acids and their abbreviations follow conventional usage. See Immunology—A Synthesis (2nd Edition, E. S. Golub and D. R. Gren, Eds., Sinauer Associates, Sunderland, Mass. (1991)).

As used herein, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present application described herein include “comprising,” “consisting,” and “consisting essentially of” aspects and embodiments.

As used herein, reference to “not” a value or parameter generally means and describes “other than” a value or parameter. For example, the method is not used to treat cancer of type X means the method is used to treat cancer of types other than X.

The term “about X-Y” used herein has the same meaning as “about X to about Y.”

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Treatment

The present application provides methods for treating cancers in a subject using an anti-CTLA4 antibody that specifically binds to human CTLA4. Any one of the anti-CTLA4 antibodies (including full-length antibodies and antigen-binding fragments thereof) in Section III “Anti-CTLA4 Antibodies” may be used in the methods described herein.

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue 1108, wherein the numbering of the amino acid residues is according to SEQ ID NO: 108, and wherein the cancer is resistant or refractory to a prior therapy, wherein the prior therapy is an inhibitor of CTLA4, PD-1, or a PD-1 ligand

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue 1108, wherein the numbering of the amino acid residues is according to SEQ ID NO: 108, and wherein the cancer is resistant or refractory to a different anti-CTLA4 antibody, such as ipilimumab.

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue 1108, wherein the numbering of the amino acid residues is according to SEQ ID NO: 108, and wherein the cancer is resistant or refractory to an inhibitor of PD-1 or a PD-1 ligand (e.g., PD-L1 or PD-L2).

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue 1108, wherein the numbering of the amino acid residues is according to SEQ ID NO: 108, and wherein the cancer is resistant or refractory to an anti-PD-1 antibody.

In some embodiments, there is provided a method of treating a cancer in a subject, wherein the cancer is resistant or refractory to an inhibitor of CTLA-4, PD-1 or a PD-1 ligand (e.g., PD-L1 or PD-L2), comprising administering to the subject an effective amount of an anti-CTLA4 antibody, wherein the antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the cancer is resistant or refractory to an anti-PD-1 antibody. In some embodiments, the cancer is resistant or refractory to a different anti-CTLA4 antibody, such as ipilimumab. In some embodiments, the cancer is resistant or refractory to an anti-PD-L1 antibody. In some embodiments, the cancer is a solid cancer, such as advanced-stage and/or metastatic cancer. In some embodiments, the cancer is urothelial carcinoma. In some embodiments, the antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some embodiments, the antibody comprises a human IgG1 Fc region, such as a wildtype IgG1 Fc region or a variant that has enhanced ADCC activity. In some embodiments, the antibody is TY21580.

In some embodiments, there is provided a method of treating a cancer in a subject, comprising administering to the subject an effective amount of an anti-CTLA4 antibody, wherein the antibody specifically binds to an epitope comprising amino acid residues Y105 and L106 of human CTLA4 but does not comprise residue I108, wherein the numbering of the amino acid residues is according to SEQ ID NO: 108, and wherein the anti-CTLA4 antibody is administered at a dose of at least about 6 mg/kg (e.g., 6 mg/kg or 10 mg/kg). In some embodiments, the anti-CTLA4 antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some embodiments, the antibody comprises a human IgG1 Fc region, such as a wildtype IgG1 Fc region or a variant that has enhanced ADCC activity. In some embodiments, the antibody is TY21580. In some embodiments, the anti-CTLA4 antibody is administered about once every three weeks. In some embodiments, the anti-CTLA4 antibody is administered intravenously. In any of the foregoing embodiments, the anti-CTLA4 antibody can be administered as a monotherapy.

In some embodiments, there is provided a method of treating a cancer in a subject, comprising administering to the subject: (a) an effective amount of an anti-CTLA4 antibody, wherein the antibody specifically binds to an epitope comprising amino acid residues Y105 and L106 of human CTLA4 but does not comprise residue 1108, wherein the numbering of the amino acid residues is according to SEQ ID NO: 108, and (b) an effective amount of an anti-PD-1 antibody. Exemplary anti-PD-1 antibodies include, but are not limited to, 2E5 (Cstone Pharmaceuticals), tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), PF-06801591, cemiplimab (REGN-2810, REGEN2810), dostarlimab (TSR-042, ANB011), pidilizumab (CT-011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, CCX-4503, biosimilars thereof, and derivatives thereof. In some embodiments, the antibodies that compete with any of these art-recognized antibodies for binding to PD-1 also can be used. In some embodiments, the anti-PD-1 antibody is 2E5. 2E5 and related anti-PD-1 antibodies have been described, for example, in CN107840887A, which is incorporated herein by reference in its entirety. In some embodiments, the anti-PD1 antibody is toripalimab. Toripalimab and related anti-PD-1 antibodies have been described, for example, in U.S. Ser. No. 10/066,013B2, which is incorporated herein by reference in its entirety.

In some embodiments, the anti-PD-1 antibody is toripalimab, a biosimilar thereof, or a derivative thereof. In some embodiments, the anti-PD-1 antibody comprises a VH and a VL, wherein the VH comprises a CDR-H1 comprising the amino acid sequence of DYEMH (SEQ ID NO: 109), a CDR-H2 comprising the amino acid sequence of VIESETGGTAYNQKFKG (SEQ ID NO: 110), and a CDR-H3 comprising the amino acid sequence of EGITTVATTYYWYFDV (SEQ ID NO; 111), and wherein the VL comprises a CDR-L1 comprising the amino acid sequence of RSSQSIVHSNGNTYLE (SEQ ID NO: 112), a CDR-L2 comprising the amino acid sequence of KVSNRFS (SEQ ID NO: 113), and a CDR-L3 comprising the amino acid sequence of FQGSHVPLT (SEQ ID NO: 114). In some embodiments, the anti-PD-1 antibody comprises a VH comprising the amino acid sequence of QGQLVQSGAEVKKPGASVKVSCKASGYTFTDYEMHWVRQAPIHGLEWIGVIESETGGT AYNQKFKGRVTITADKSTSTAYMELSSLRSEDTAVYYCAREGITTVATTYYWYFDVWG QGTTVTVSS (SEQ ID NO: 115), and/or a VL comprising the amino acid sequence of DVVMTQSPLSLPVTLGQPASISCRSSQSIVHSNGNTYLEWYLQKPGQSPQLLIYKVSNRF SGVPDRFSGSGSGTDFTLKISRVEAEDVGVYYCFQGSHVPLTFGQGTKLEIK (SEQ ID NO: 116). In some embodiments, an effective amount of the anti-PD-1 antibody is administered at about 1 mg/kg to about 10 mg/kg. In some embodiments, the administered dose of the anti-PD1 antibody is 1 mg/kg. In some embodiments, the administered dose of the anti-PD1 antibody is 5 mg/kg. In some embodiments, the administered dose of the anti-PD1 antibody is 10 mg/kg. In some embodiments, the anti-PD-1 antibody is administered at a dose of about 240 mg. In some embodiments, the anti-PD-1 antibody is administered intravenously. In some embodiments, the anti-PD-1 antibody is administered every three weeks. In some embodiments, the cancer is selected from the group consisting of renal cell carcinoma, non-small cell lung cancer, hepatocellular carcinoma, and microsatellite instability-high or mismatch repair deficient cancers.

In some embodiments where the anti-CTLA4 antibody is administered in combination with an anti-PD-1 antibody, the anti-CTLA4 antibody comprises: (a) a heavy chain variable region comprising an HVR-H1 comprising the amino acid sequence of SEQ ID NO: 23, an HVR-H2 comprising the amino acid sequence of SEQ ID NO: 35, and an HVR-H3 comprising the amino acid sequence of SEQ ID NO: 45, and/or a light chain variable region comprising an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the antibody comprises a heavy chain variable region comprising the amino acid sequence of SEQ ID NO: 87 or an amino acid sequence having at least 90% sequence identity to the amino acid sequence of SEQ ID NO: 87, and/or a light chain variable region comprising the amino acid sequence of SEQ ID NO: 100 or an amino acid sequence having at least 90% sequence identity to the amino acid sequence of SEQ ID NO: 100. In some embodiments, the antibody comprises a human IgG1 Fc region, such as a wildtype IgG1 Fc region or a variant that has enhanced ADCC activity. In some embodiments, the antibody is TY21580. In some embodiments, the anti-CTLA4 antibody is administered at a dose of at least about 1 mg/kg (e.g., 2 mg/kg, 3 mg/kg or 6 mg/kg). In some embodiments, the anti-CTLA4 antibody is administered about once every three weeks. In other embodiments, the anti-CTLA4 antibody is administered about once every six weeks. In some embodiments, the anti-CTLA4 antibody is administered intravenously. In some embodiments, the cancer is resistant or refractory to an inhibitor of CTLA-4, PD-1 or a PD-1 ligand (e.g., PD-L1 or PD-L2). In some embodiments, the cancer is a solid cancer, such as advanced-stage and/or metastatic cancer. In some embodiments, the cancer is urothelial carcinoma. Cancer treatments can be evaluated by, e.g., tumor regression, tumor weight or size shrinkage, time to progression, duration of survival, progression free survival, overall response rate, duration of response, quality of life, protein expression and/or activity. Approaches to determining efficacy of therapy can be employed, including for example, measurement of response through radiological imaging.

The anti-CTLA4 antibodies and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The antibodies and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific antibody being used, the rate of absorption desired, specific formulation or dosage form used, type or severity of the disorder being treated, the specific site of action, and conditions of the patient, and can be readily selected by a person skilled in the art. In some embodiments, the anti-CTLA4 antibody is administered intravenously.

The effective amount of the anti-CTLA4 antibody may be administered in a single dose or in multiple doses. For methods that comprises administration of the anti-CTLA4 antibody in multiple doses, exemplary dosing frequencies include, but are not limited to, weekly, weekly without break, weekly for two out of three weeks, weekly for three out of four weeks, once every three weeks, once every two weeks, monthly, every six months, yearly, etc. In some embodiments, the anti-CTLA4 antibody is administered about weekly, once every 2 weeks, once every 3 weeks, once every 6 weeks, or once every 12 weeks. In some embodiments, the intervals between each administration are less than about any of 3 years, 2 years, 12 months, 11 months, 10 months, 9 months, 8 months, 7 months, 6 months, 5 months, 4 months, 3 months, 2 months, 1 month, 4 weeks, 3 weeks, 2 weeks, or 1 week. In some embodiments, the intervals between each administration are more than about any of 1 week, 2 weeks, 3 weeks, 4 weeks, 1 month, 2 months, 3 months, 4 months, 5 months, 6 months, 7 months, 8 months, 9 months, 10 months, 11 months, 12 months, 2 years, or 3 years. In some embodiments, there is no break in the dosing schedule.

In some embodiments, the anti-CTLA4 antibody is administered at a low frequency, for example, any one of no more frequent than once per week, once every other week, once per three weeks, once per month, once per 2 months, once per 3 months, once per 4 months, once per 5 months, once per 6 months, once per 7 months, once per 8 months, once per 9 months, once per 10 months, once per 11 months, once per year, or less. In some embodiments, the anti-CTLA4 antibody is administered in a single dose. In some embodiments, the anti-CTLA4 antibody is administered about once every three weeks.

In some embodiments, the anti-CTLA4 antibody is administered for 2 or more cycles, such as about any one of 2, 3, 4, 5, 6, 7, 8, 9, 10, 11, 12, or more cycles. In some embodiments, the anti-CTLA4 antibody is administered for at least 4 cycles.

The anti-CTLA4 antibodies can be administered to patients as monotherapies or in combination with other therapeutic agents (e.g., an anti-PD-1 antibody) at doses that achieve high levels of receptor (CTLA-4) occupancy, and hence are efficacious while at the same time having minimal side effects. Hence, the anti-CTLA4 antibodies of the disclosure show improved therapeutic indexes relative to anti-CTLA4 antibodies such as Ipilimumab. For instance, in one embodiment, wherein the antibody comprises a heavy chain variable region comprising the amino acid sequence of SEQ ID NO: 87 and light chain variable region comprising the amino acid sequence of SEQ ID NO: 100, the anti-CTLA4 antibody can be administered as a single dose (either as a monotherapy or in combination with one or more therapeutic agents) that achieves greater than 50% receptor occupancy three weeks or even six weeks following administration. In some such embodiments, the anti-CTLA4 antibody can be administered as a single dose that achieves greater than 60% receptor occupancy three weeks following administration. In other such embodiments, the anti-CTLA4 antibody can be administered as a single dose that achieves greater than 70% receptor occupancy three weeks following administration. In other such embodiments, the anti-CTLA4 antibody can be administered as a single dose that achieves greater than 80% receptor occupancy three weeks following administration. In other such embodiments, the anti-CTLA4 antibody can be administered as a single dose that achieves from about 50% to about 80% receptor occupancy three weeks following administration. In other such embodiments, the anti-CTLA4 antibody can be administered as a single dose that achieves from about 60% to about 75% receptor occupancy three weeks following administration. In other such embodiments, the anti-CTLA4 antibody can be administered as a single dose that achieves greater than 60% receptor occupancy six weeks following administration. In other such embodiments, the anti-CTLA4 antibody can be administered as a single dose that achieves greater than 70% receptor occupancy six weeks following administration. In other such embodiments, the anti-CTLA4 antibody can be administered as a single dose that achieves from about 50% to about 70% receptor occupancy six weeks following administration. In any of the foregoing embodiments, the anti-CTLA4 antibody can be administered in combination with an anti-PD-1 antibody, as disclosed herein.

In one embodiment, wherein the antibody comprises a heavy chain variable region comprising the amino acid sequence of SEQ ID NO: 87 and light chain variable region comprising the amino acid sequence of SEQ ID NO: 100, the antibody can be administered to the subject at dose of from about 1 mg/kg to about 10 mg/kg or from about 2 mg/kg to about 5 mg/kg. In some such embodiments, the anti-CTLA4 antibody can be administered to the subject at a dose of about 3 mg/kg. In some embodiments, the anti-CTLA4 antibody can be administered to the subject once every three weeks. In other embodiments, the anti-CTLA4 antibody can be administered to the subject once every six weeks. In particular embodiments, the antibody TY21580 can be administered to a patient at a dose of about 3 mg/kg once every three weeks or once every six weeks.

In some embodiments, the treatment comprises an initial phase and a subsequent maintenance phase. In some embodiments, the anti-CTLA4 antibody is administered less frequently in the maintenance phase than in the initial phase. In some embodiments, the anti-CTLA4 antibody is administered at the same frequently in the maintenance phase as in the initial phase. In some embodiments, the treatment comprises an initial phase wherein the anti-CTLA4 antibody is administered about once every three weeks for at least 4 cycles, and a maintenance phase wherein the anti-CTLA4 antibody is administered about once every 4 weeks to once every 12 weeks, such as once every 4 weeks, once every 6 weeks, once every 8 weeks, once every 10 weeks, or once every 12 weeks. In some embodiments, the dosing frequency in the maintenance phase is adjusted depending on one or more biomarkers, such as T_{reg} cells, CD8+Tₑₘ cells, CD4+Tₑₘ cells, a ratio of CD8+Tₑₘ cells to T_{reg} cells, a ratio of CD4+Tₑₘ cells to T_{reg} cells, and/or NK cells. For example, if the subject shows an increase in the ratio of CD8+Tₑₘ cells to T_{reg} cells after receiving the anti-CTLA4 antibody, the subject may be further administered an anti-CTLA4 antibody at about every 4 weeks.

The administration of the anti-CTLA4 antibody can be extended over an extended period of time, such as from about a week to about a month, from about a month to about a year, from about a year to about several years. In some embodiments, the anti-CTLA4 antibody is administered over a period of at least any of about 1 week, 2 weeks, 3 weeks, 4 weeks, 5 weeks, 1 month, 2 months, 3 months, 4 months, 5 months, 6 months, 7 months, 8 months, 9 months, 10 months, 11 months, 12 months, 1 year, 2 years, 3 years, 4 years, or more.

The methods described herein are useful for treating a variety of cancers. In some embodiments, the cancer is a solid cancer. In some embodiments, the cancer is a liquid cancer. A variety of cancers where CTLA4 is implicated, whether malignant or benign and whether primary or secondary, may be treated or prevented with a method provided by the disclosure. Exemplary cancers include, but are not limited to, liver cancer, a cancer of the digestive system (e.g., colon cancer, colorectal cancer), lung cancer, bone cancer, heart cancer, brain cancer, kidney cancer, bladder cancer, a hematological cancer (e.g., leukemia), skin cancer, breast cancer, thyroid cancer, pancreatic cancer, a head and/or neck cancer, an eye-related cancer, a male reproductive system cancer (e.g., prostate cancer, testicular cancer), or a female reproductive system cancer (e.g., uterine cancer, cervical cancer). In some embodiments, the cancer is kidney cancer, such as renal cell carcinoma, or urothelial carcinoma. In some embodiments, the cancer is a cold tumor. In some embodiments, the cancer is resistant or refractory to one or more prior therapies, such as immunotherapies, including immune checkpoint inhibitor(s). In some embodiments, the cancer is a tumor that T cells cannot penetrate because the tumor has not been recognized by the immune system, or provoked an immune response.

In particular embodiments, the anti-CTLA4 antibodies of the disclosure can be used to treat Kaposi's sarcoma. In some such embodiments, the anti-CTLA4 antibody is administered as a monotherapy. In other such embodiments, the anti-CTLA4 antibody with an additional therapeutic reagent, in particular an anti-PD-1 antibody. In any of the foregoing embodiments, the the anti-CTLA4 antibody can comprise a heavy chain variable region comprising the amino acid sequence of SEQ ID NO: 87 and light chain variable region comprising the amino acid sequence of SEQ ID NO: 100. In some such embodiments, the anti-CTLA4 antibody is TY21580.

In particular embodiments, the anti-CTLA4 antibodies of the disclosure can be used to treat head and neck squamous cell carcinoma (HNSCC). In some such embodiments, the anti-CTLA4 antibody is administered as a monotherapy. In other such embodiments, the anti-CTLA4 antibody with an additional therapeutic reagent, in particular an anti-PD-1 antibody. In any of the foregoing embodiments, the anti-CTLA4 antibody can comprise a heavy chain variable region comprising the amino acid sequence of SEQ ID NO: 87 and light chain variable region comprising the amino acid sequence of SEQ ID NO: 100. In some such embodiments, the anti-CTLA4 antibody is TY21580.

In particular embodiments, the anti-CTLA4 antibodies of the disclosure can be used to treat pancreatic cancer. In some such embodiments, the anti-CTLA4 antibody is administered as a monotherapy. In other such embodiments, the anti-CTLA4 antibody with an additional therapeutic reagent, in particular an anti-PD-1 antibody. In any of the foregoing embodiments, the anti-CTLA4 antibody can comprise a heavy chain variable region comprising the amino acid sequence of SEQ ID NO: 87 and light chain variable region comprising the amino acid sequence of SEQ ID NO: 100. In some such embodiments, the anti-CTLA4 antibody is TY21580.

In particular embodiments, the anti-CTLA4 antibodies of the disclosure can be used to treat ovarian cancer. In some such embodiments, the anti-CTLA4 antibody is administered as a monotherapy. In other such embodiments, the anti-CTLA4 antibody with an additional therapeutic reagent, in particular an anti-PD-1 antibody. In any of the foregoing embodiments, the anti-CTLA4 antibody can comprise a heavy chain variable region comprising the amino acid sequence of SEQ ID NO: 87 and light chain variable region comprising the amino acid sequence of SEQ ID NO: 100. In some such embodiments, the anti-CTLA4 antibody is TY21580.

In some embodiments, the subject has been previously treated with a prior therapy. In some embodiments, the subject has previously received any one of 1, 2, 3, 4, or more prior therapies. In some embodiments, the subject has exhausted all other available therapies. In some embodiments, the subject is unresponsive or resistant to a prior therapy. In some embodiments, the subject has disease reoccurrence subsequent to a prior therapy. In some embodiments, the subject is refractory to a prior therapy. In some embodiments, the subject has failed a prior therapy within about 1 year, 6 months, 3 months or less. In some embodiments, the subject has not previously received a prior therapy.

In some embodiments, the subject has been previously treated with a standard therapy for the cancer. In some embodiments, the subject is unresponsive or resistant to a standard therapy. In some embodiments, the subject has disease reoccurrence subsequent to a standard therapy. In some embodiments, the subject is refractory to a standard therapy. In some embodiments, the subject has failed a standard therapy within about 1 year, 6 months, 3 months or less. In some embodiments, the subject has not previously received a standard therapy. In some embodiments, the subject has refused or is ineligible for a standard therapy.

In some embodiments, the prior therapy (e.g., standard therapy) is selected from the group consisting of viral gene therapy, immunotherapy, targeted therapy, radiation therapy, and chemotherapy. In some embodiments, the prior therapy is an immune checkpoint inhibitor. In some embodiments, the prior therapy is an inhibitor of CTLA4, PD-1, or a PD-1 ligand (e.g., PD-L1 or PD-L2). In some embodiments, the prior therapy is an inhibitor of CTLA4, such as an anti-CTLA4 antibody that is different from the anti-CTLA4 antibodies described herein. In some embodiments, the prior therapy is ipilimumab.

In some embodiments, the prior therapy is an inhibitor of PD-1 or a PD-1 ligand, including a PD-1 binding antagonist, a PDL1 binding antagonist and a PDL2 binding antagonist. Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the inhibitor of PD-1 is a molecule that inhibits the binding of PD-1 to its ligand binding partners. In some embodiments, the inhibitor of a PD-1 ligand is an inhibitor of PD-L1 and/or PD-L2. In some embodiments, the inhibitor of PD-L1 is a molecule that inhibits the binding of PDL1 to its binding partners. In some embodiments, a PD-L2 binding partner is PD-1 and/or B7-1. In some embodiments, the inhibitor of a PD-1 ligand is a molecule that inhibits the binding of PD-L2 to its binding partners. In some embodiments, a PD-L2 binding partner is PD-1. The inhibitor may be an antibody, an antigen binding fragment thereof, an immunoadhesin, a fusion protein, or oligopeptide.

In some embodiments, the inhibitor of PD-1 is an anti-PD-1 antibody (e.g., a human antibody, a humanized antibody, or a chimeric antibody). In some embodiments, the anti-PD-1 antibody is 2E5 (Cstone Pharmaceuticals), tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), PF-06801591, cemiplimab (REGN-2810, REGEN2810), dostarlimab (TSR-042, ANB011), pidilizumab (CT-011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, CCX-4503, biosimilars thereof, or derivatives thereof. In some embodiments, the anti-PD-1 antibody is selected from the group consisting of nivolumab, and CT-011. In some embodiments, the inhibitor of PD-1 is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the inhibitor of PD-1 is AMP-224. In some embodiments, the anti-PD-1 antibody is nivolumab (CAS Registry Number: 946414-94-4). Nivolumab, also known as MDX-1106-04, MDX-1106, ONO-4538, BMS-936558, and OPDIVO®, is an anti-PD-1 antibody described in WO2006/121168. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

In some embodiments, the inhibitor of PD-L1 is anti-PD-L1 antibody. In some embodiments, the inhibitor of PD-L1 is selected from the group consisting of YW243.55.S70, MPDL3280A, MDX-1105, and MEDI4736. MDX-1105, also known as BMS-936559, is an anti-PD-L1 antibody described in WO2007/005874. Antibody YW243.55.S70 (heavy and light chain variable region sequences shown in SEQ ID Nos. 20 and 21, respectively) is an anti-PD-L1 described in WO 2010/077634 A1. MEDI4736 is an anti-PD-L1 antibody described in WO2011/066389 and US2013/034559. Examples of anti-PD-L1 antibodies useful for the methods of this application, and methods for making thereof are described in PCT patent application WO 2010/077634 A1 and U.S. Pat. No. 8,217,149, which are incorporated herein by reference.

Prior therapies (e.g., standard therapies) also encompass surgery to remove a tumor and radiation therapy. Exemplary radiation therapies include, but are not limited to, ionizing (electromagnetic) radiotherapy (e.g., X-rays or gamma rays) and particle beam radiation therapy (e.g., high linear energy radiation). The source of radiation can be external or internal to the subject.

The methods described herein are useful for various aspects of cancer treatment. In some embodiments, there is provided a method of inhibiting cell proliferation (such as tumor growth) in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) cell proliferation is inhibited.

In some embodiments, there is provided a method of inhibiting tumor metastasis in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) metastasis is inhibited.

In some embodiments, there is provided a method of reducing (such as eradicating) pre-existing tumor metastasis (such as metastasis to the lymph node) in an individual, comprising administering to the individual an effective amount of any one of the anti-CTLA4 antibodies described herein. In some embodiments, at least about 10% (including for example at least about any of 20%, 30%, 40%, 60%, 70%, 80%, 90%, 95% or more) metastasis is reduced.

In some embodiments, there is provided a method of reducing incidence or burden of preexisting tumor metastasis (such as metastasis to the lymph node) in an individual, comprising administering to the individual an effective amount of any one of the anti-CTLA4 antibodies described herein.

In some embodiments, there is provided a method of reducing tumor size in an individual, comprising administering to the individual an effective amount of any one of the anti-CTLA4 antibodies described herein. In some embodiments, the method reduces tumor size by at least about 10% (including for example at least about any of 20%, 30%, 40%, 60%, 70%, 80%, 90%, 95% or more).

In some embodiments, there is provided a method of prolonging time to disease progression of cancer in an individual, comprising administering to the individual an effective amount of any one of the anti-CTLA4 antibodies described herein. In some embodiments, the method prolongs the time to disease progression by at least any of 1, 2, 3, 4, 5, 6, 7, 8, 9, 10, 11, 12, 16, 20, 24, 28, 32, 36, or more weeks.

In some embodiments, there is provided a method of prolonging survival (e.g., overall survival or progression-free survival) of an individual having cancer, comprising administering to the individual an effective amount of any one of the anti-CTLA4 antibodies described herein. In some embodiments, the method prolongs the survival of the individual by at least any of 1, 2, 3, 4, 5, 6, 7, 8, 9, 10, 11, 12, 18, or 24 months.

In some embodiments, there is provided a method of alleviating one or more symptoms in an individual having cancer, comprising administering to the individual an effective amount of any one of the anti-CTLA4 antibodies described herein.

In some embodiments, there is provided a method of improving the quality of life in an individual having cancer, comprising administering to the individual an effective amount of any one of the anti-CTLA4 antibodies described herein.

The anti-CTLA4 antibody may be administered alone as monotherapy, or administered in combination with one or more additional therapeutic agents or therapies. In some embodiment, anti-CTLA4 antibody is administered in combination with one or more additional therapeutic agents for separate, sequential or simultaneous administration. The term “additional therapeutic agent” refers to any therapeutic agent other than an anti-CTLA4 antibody provided by the disclosure. In some embodiments, there is provided a combination therapy for treating cancer in a subject, which comprises administering to the subject a therapeutically effective amount of an anti-CTLA4 antibody described herein in combination with one or more additional therapeutic agents.

In some embodiments, anti-CTLA4 antibody is administered in combination with one or more additional therapeutic agents comprising chemotherapeutic agents, immunotherapeutic agents, and/or hormone therapeutic agents. In some embodiments, the one or more additional therapeutic agents are selected from the group consisting of selected from the group consisting of viral gene therapy, immune checkpoint inhibitors, targeted therapies, radiation therapies, and chemotherapies. In particular embodiments, at least one of the additional therapeutic agent is an anti-PD-1 antibody, as described herein.

### III. Anti-CTLA4 Antibodies

The method described herein comprise administration of an anti-CTLA4 antibody that specifically binds to human CTLA4, including CTLA4 antibodies, antigen-binding fragments of the CTLA4 antibodies, and derivatives of the CTLA4 antibodies. Exemplary anti-CTLA4 antibodies have been described, for example, in International Publication No. WO2019149281A1, which is incorporated herein by reference in its entirety.

In some embodiments, the anti-CTLA4 antibody is any one of the antibodies described herein, including antibodies described with reference to specific amino acid sequences of HVRs, variable regions (VL, VH), and light and heavy chains (e.g., IgG1, IgG2, IgG4). In some embodiments, the antibodies are human antibodies. In some embodiments, the antibodies are humanized antibodies and/or chimeric antibodies. In some embodiments, the anti-CTLA4 antibody binds to human CTLA4, and have at least one (e.g., at least one, at least two, at least three, at least four, at least five, at least six, at least seven, at least eight, or all nine) of the following functional properties: (a) bind to human, cynomolgus monkey, mouse, rat, and/or dog CTLA4 with a K_{D} of 500 nM or less; (b) have antagonist activity on human CTLA4; (c) do not bind to human PD-1, PD-L1, PD-L2, LAG3, TIM3, B7-H3, CD95, CD120a, OX40, CD40, BTLA, VISTA, ICOS, and/or B7-H4 at concentration up to 100 nM; (d) are cross-reactive with monkey, mouse, rat, and/or dog CTLA4; (e) induces ADCC effects (e.g., on Tregs); (f) activates human PBMCs (e.g., stimulates secretion of IL-2 and/or IFNT); (g) are capable of inhibiting tumor cell growth; (h) have therapeutic effect on a cancer; and (i) block binding of human CTLA4 to human CD80 and/or human CD86. In some embodiments, the anti-CTLA4 antibodies described herein have lower activity in blocking binding of CD80 and/or CD86 to human CTLA4 as compared to ipilimumab in an assay wherein either when human CD80 and/or CD86 are immobilized (or plate bound) or when human CTLA4 protein is present on cell surface. In some embodiments, the anti-CTLA4 antibodies described herein deplete Treg cells selectively in tumor microenvironment as compared to Treg depletions in PBMC or spleen. In some embodiments, the anti-CTLA4 antibodies described herein have higher Treg depletion activity in tumor microenvironment as compared to ipilimumab. Also provided herein are one or more anti-CTLA4 antibodies or antigen-binding fragments that cross-compete for binding to human CTLA4 with one or more of the antibodies or antigen-binding fragments described herein.

In some embodiments, the antibodies or antigen-binding fragments bind to human, cynomolgus monkey, mouse, rat, and/or dog CTLA4 with a K_{D} of about 500 nM or less (e.g., about 500 nM or less, about 450 nM or less, about 400 nM or less, about 350 nM or less, about 300 nM or less, about 250 nM or less, about 200 nM or less, about 150 nM or less, about 100 nM or less, about 90 nM or less, about 80 nM or less, about 70 nM or less, about 60 nM or less, about 50 nM or less, about 40 nM or less, about 30 nM or less, about 25 nM or less, about 20 nM or less, about 10 nM or less, about 1 nM or less, about 0.1 nM or less, etc.) In some embodiments, the antibodies or antigen-binding fragments bind to human, cynomolgus monkey, mouse, rat, and/or dog CTLA4 with a K_{D} of about 350 nM or less. In some embodiments, the antibodies or antigen-binding fragments bind to human CTLA4 with a K_{D} of about 100 nM or less. In some embodiments, the antibodies or antigen-binding fragments bind to human CTLA4 with a K_{D} of about 50 nM or less. In some embodiments, the antibodies or antigen-binding fragments bind to human CTLA4 with a K_{D} of about 10 nM or less. Methods of measuring the K_{D} of an antibody or antigen-binding fragment may be carried out using any method known in the art, including for example, by surface plasmon resonance, an ELISA, isothermal titration calorimetry, a filter binding assay, an EMSA, etc. In some embodiments, the K_{D} is measured by surface plasmon resonance or an ELISA (see e.g., Example 3 below).

In some embodiments, the antibodies or antigen-binding fragments described herein have antagonist activity on human CTLA4. In some embodiments, the antibodies or antigen-binding fragments repress one or more activities of human CTLA4 when a cell (e.g., a human cell) expressing human CTLA4 is contacted by the antibody or antigen binding fragment (e.g., CTLA4 blockade as measured by an increase in a reporter gene signal using a CLA4 blockage reporter gene assay).

In some embodiments, the antibodies or antigen-binding fragments are cross-reactive with monkey (e.g., cynomolgus monkey), mouse, rat, and/or dog CTLA4. In some embodiments, the antibodies or antigen-binding fragments are cross-reactive with monkey CTLA4. In some embodiments, the antibodies or antigen-binding fragments are cross-reactive with mouse CTLA4. In some embodiments, the antibodies or antigen-binding fragments are cross-reactive with rat CTLA4. In some embodiments, the antibodies or antigen-binding fragments are cross-reactive with dog CTLA4. In some embodiments, the antibodies or antigen binding fragments are cross reactive with monkey and mouse CTLA4; monkey and rat CTLA4; monkey and dog CTLA4; mouse and rat CTLA4; mouse and dog CTLA4; rat and dog CTLA4; monkey, mouse, and rat CTLA4; monkey, mouse, and dog CTLA4; monkey, rat, and dog CTLA4; mouse, rat, and dog CTLA4; or monkey, mouse, rat, and dog CTLA4. In some embodiments, the antibodies or antigen binding fragments are cross-reactive if the antibodies or antigen-binding fragments binds to a non-human CTLA4 molecule with a K_{D} less than about 500 nM (e.g., less than about 1 nM, less than about 10 nM, less than about 25 nM, less than about 50 nM, less than about 75 nM, less than about 100 nM, less than about 150 nM, less than about 200 nM, less than about 250 nM, less than about 300 nM, less than about 350 nM, etc.). Methods of measuring antibody cross-reactivity are known in the art, including, without limitation, surface plasmon resonance, an ELISA, isothermal titration calorimetry, a filter binding assay, an EMSA, etc. In some embodiments, the cross-reactivity is measured by ELISA.

In some embodiments, the antibodies induce ADCC effects against a CTLA4 expressing cell (e.g., against CTLA4-expressing human cells such as Tregs) after the antibody binds to the cell-expressed CTLA4. Methods of measuring ADCC effects (e.g., in vitro methods) are known in the art. In some embodiments, the antibodies induce ADCC effects by more than about 10% (e.g., induce ADCC by more than about 10%, more than about 15%, more than about 20%, more than about 25%, more than about 30%, more than about 35%, more than about 40%, etc.) relative to a control (e.g., an isotype control or ipilimumab).

In some embodiments, the antibodies or antigen-binding fragments are capable of inhibiting tumor cell growth and/or proliferation. In some embodiments, the tumor cell growth and/or proliferation is inhibited by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) when contacted with the antibodies or antigen-binding fragments relative to corresponding tumor cells not contacted with the antibodies or antigen-binding fragments (or relative to corresponding tumor cells contacted with an isotype control antibody). In some embodiments, the antibodies or antigen-binding fragments are capable of reducing tumor volume in a subject when the subject is administered the antibodies or antigen-binding fragments. In some embodiments, the antibodies or antigen-binding fragments are capable of reducing tumor volume in a subject by at least about 5% (e.g., at least about 5%, at least about 10%, at least about 20%, at least about 30%, at least about 40%, at least about 50%, at least about 60%, at least about 70%, at least about 80%, at least about 90%, or at least about 99%) relative to the initial tumor volume in the subject (e.g., prior to administration of the antibodies or antigen-binding fragments; as compared to a corresponding tumor in a subject administered an isotype control antibody). Methods of monitoring tumor cell growth/proliferation, tumor volume, and/or tumor inhibition are known in the art.

In some embodiments, the antibodies or antigen-binding fragments have therapeutic effect on a cancer. In some embodiments, the antibodies or antigen-binding fragments reduce one or more signs or symptoms of a cancer. In some embodiments, a subject suffering from a cancer goes into partial or complete remission when administered the antibodies or antigen-binding fragments.

In another aspect, the disclosure provides isolated antibodies that compete or cross-compete for binding to human CTLA4 with any of the illustrative antibodies of the disclosure, such as TY21585, TY21586, TY21587, TY21588, TY21589, TY21580, TY21591, TY21686, TY21687, TY21689, TY21680, TY21691, and/or TY21692. In a particular embodiment, the present application provides isolated antibodies that compete or cross-compete for binding to the same epitope on the human CTLA4 with any of the illustrative antibodies of the disclosure. The ability of an antibody to compete or cross-compete for binding with another antibody can be determined using standard binding assays known in the art, such as BIAcore analysis, ELISA assays, or flow cytometry. For example, one can allow an illustrative antibody of the disclosure to bind to human CTLA4 under saturating conditions and then measure the ability of the test antibody to bind to the CTLA4. If the test antibody is able to bind to the CTLA4 at the same time as the illustrative antibody, then the test antibody binds to a different epitope as the illustrative antibody. However, if the test antibody is not able to bind to the CTLA4 at the same time, then the test antibody binds to the same epitope, an overlapping epitope, or an epitope that is in close proximity to the epitope bound by the illustrative antibody. This experiment can be performed using various methods, such as ELISA, RIA, FACS or surface plasmon resonance.

In some embodiments, the antibodies or antigen-binding fragments block the binding between CTLA4 and one or more of its binding partners (e.g., human CTLA4 and human CD80, human CTLA4 and human CD86). In some embodiments, the antibodies or antigen-binding fragments block the binding between CTLA4 and its ligand in vitro. In some embodiments, the antibody or antigen-binding fragment has a half maximal inhibitory concentration (IC₅₀) of about 500 nM or less (e.g., about 500 nM or less, about 400 nM or less, about 300 nM or less, about 200 nM or less, about 100 nM or less, about 50 nM or less, about 25 nM or less, about 10 nM or less, about 1 nM or less, etc.) for blocking binding of CTLA4 to CD80 and/or CD86. In some embodiments, the antibody or antigen-binding fragment has a half maximal inhibitory concentration (IC₅₀) of about 100 nM or less for blocking binding of CTLA4 to CD80 and/or CD86. In some embodiments, the antibody or antigen-binding fragment completely blocks binding of human CTLA4 to CD80 and/or CD86 when provided at a concentration of about 100 nM or greater (e.g., about 100 nM or greater, about 500 nM or greater, about 1 μM or greater, about 10 μM or greater, etc.). As used herein, the term “complete blocking” or “completely blocks” refers to the antibody or antigen-binding fragment's ability to reduce binding between a first protein and a second protein by at least about 80% (e.g., at least about 80%, at least about 85%, at least about 90%, at least about 95%, at least about 99%, etc.). Methods of measuring the ability of an antibody or antigen-binding fragment to block binding of a first protein (e.g., human CTLA4) and a second protein (e.g., human CD80 or human CD86) are known in the art, including, without limitation, via BIAcore analysis, ELISA assays, and flow cytometry. In some embodiments, the anti-CTLA4 antibodies described herein have lower activity in blocking ligand binding than ipilimumab.

In some embodiments, the anti-CTLA4 antibody binds human CTLA4 with a K_{D} of 1000 nM or less (e.g., 50 nM or less, 10 nM or less) as measured by surface plasmon resonance. In some embodiments, the antibody is cross-reactive with at least one non-human species selected from cynomolgus monkey, mouse, rat, and dog.

In some embodiments, the anti-CTLA4 antibody specifically binds to an epitope similar to a ligand binding site of human CTLA4. In some embodiments, the antibody specifically binds to an epitope similar to CD80 binding site of human CTLA4. In some embodiments, the antibody specifically binds to an epitope similar to CD86 binding site of human CTLA4. In some embodiments, the antibody specifically binds to an epitope comprising one or more amino acid residues in a ligand binding site (e.g., CD80 and/or CD86 binding site) of human CTLA4. In some embodiments, the antibody specifically binds to an epitope on human CTLA4 that is different from the epitope of ipilimumab. In some embodiments, the epitope does not comprise amino acid residues in the CC′ loop motif of human CTLA4. In some embodiments, the epitope does not comprise amino acid residue L106 or 1108 of human CTLA4. In some embodiments, the antibody specifically binds to an epitope comprising amino acid residues Y105 and L106, but not 1108 of human CTLA4, wherein the numbering of the amino acid residues is according to

(SEQ ID NO: 108)
KAMHVAQPAVVLASSRGIASFVCEYASPGKATEVRVTVLRQADSQVTEV
CAATYMMGNELTFLDDSICTGTSSGNQVNLTIQGLRAMDTGLYICKVEL
MYPPPYYLGIGNGTQIYVIDPE

In some embodiments, the anti-CTLA4 antibody comprising a heavy chain variable region and a light chain variable region, a) where the heavy chain variable region comprises an HVR-H1, an HVR-H2, and an HVR-H3, where the HVR-H1 comprises an amino acid sequence according to a formula selected from: Formula (I): X1TFSX2YX3IHWV (SEQ ID NO: 1), where X1 is F or Y, X2 is D or G, and X3 is A, G, or W; Formula (II): YSIX1SGX2X3WX4WI (SEQ ID NO: 2), where X1 is S or T, X2 is H or Y, X3 is H or Y, and X4 is A, D, or S; and Formula (III): FSLSTGGVAVX1WI (SEQ ID NO: 3), where X1 is G or S; the HVR-H2 comprises an amino acid sequence according to a formula selected from: Formula (IV): IGX1IX2HSGSTYYSX3SLKSRV (SEQ ID NO: 4), where X1 is D or E, X2 is S or Y, and X3 is P or Q; Formula (V): IGX1ISPSX2GX3TX4YAQKFQGRV (SEQ ID NO: 5), where X1 is I or W, X2 is G or S, X3 is G or S, and X4 is K or N; and Formula (VI): VSX1ISGX2GX3X4TYYADSVKGRF (SEQ ID NO: 6), where X1 is A, G, or S, X2 is S or Y, X3 is G or S, and X4 is S or T; and the HVR-H3 comprises an amino acid sequence according to a formula selected from: Formula (VII): ARX1X2X3X4FDX5 (SEQ ID NO: 7), where X1 is G, R, or S, X2 is A, I, or Y, X3 is D, V, or Y, X4 is A, E, or Y, and X5 is I or Y; Formula (VIII): ARX1GX2GYFDX3 (SEQ ID NO: 8), where X1 is D or L, X2 is F or Y, and X3 is V or Y; Formula (IX): ARX1X2X3X4AX5X6FDY (SEQ ID NO: 9), where X1 is L or R, X2 is I or P, X3 is A or Y, X4 is S or T, X5 is T or Y, and X6 is A or Y; and Formula (X): ARDX1X2X3GSSGYYX4GFDX5 (SEQ ID NO: 10), where X1 is I or V, X2 is A or H, X3 is P or S, X4 is D or Y, and X5 is F or V; and/or b) where the light chain variable region comprises an HVR-L1, an HVR-L2, and an HVR-L3, where the HVR-L1 comprises an amino acid sequence according to a formula selected from: Formula (XI): RASQX1X2X3SX4LX5 (SEQ ID NO: 11), where X1 is G or S, X2 is I or V, X3 is G or S, X4 is S or Y, and X5 is A or N; Formula (XII): RASQX1VX2X3RX4LA (SEQ ID NO: 12), where X1 is S or T, X2 is F, R, or S, X3 is G or S, and X4 is F or Y; and Formula (XIII): RASX1SVDFX2GX3SFLX4 (SEQ ID NO: 13), where X1 is E or Q, X2 is D, F, H, or Y, X3 is F, I, or K, and X4 is A, D, or H; the HVR-L2 comprises an amino acid sequence according to Formula (XIV): X1ASX2X3X4X5GX6 (SEQ ID NO: 14), where X1 is A or D, X2 is N, S, or T, X3 is L or R, X4 is A, E, or Q, X5 is S or T, and X6 is I or V; and the HVR-L3 comprises an amino acid sequence according to a formula selected from: Formula (XV): YCX1X2X3X4X5X6PX7T (SEQ ID NO: 15), where X1 is E, Q, or V, X2 is H or Q, X3 is A, G, H, R, or S, X4 is D, L, S, or Y, X5 is E, G, P, Q, or S, X6 is L, T, V, or W, and X7 is F, L, P, W, or Y; Formula (XVI): YCQQX1X2X3WPPWT (SEQ ID NO: 16), where X1 is S or Y, X2 is D or Y, and X3 is Q or Y; and Formula (XVII): YCQX1YX2SSPPX3YT (SEQ ID NO: 17), where X1 is H or Q, X2 is T or V, and X3 is E or V.

In some embodiments, the antibody comprises: a) an HVR-H1 comprising an amino acid sequence selected from SEQ ID NOS: 18-29; an HVR-H2 comprising an amino acid sequence selected from SEQ ID NOS: 30-39; and an HVR-H3 comprising an amino acid sequence selected from SEQ ID NOS: 40-52; and/or b) an HVR-L1 comprising an amino acid sequence selected from SEQ ID NOS: 53-65; an HVR-L2 comprising an amino acid sequence selected from SEQ ID NOS: 66-69; and an HVR-L3 comprising an amino acid sequence selected from SEQ ID NOS: 70-81. In some embodiments, the antibody comprises one, two, three, four, five, or all six of the HVRs shown for any of the exemplary antibodies described in Table A below.

TABLE A
anti-CTLA4 HVR sequences

| Ab name: | HVR-H1 | HVR-H2 | HVR-H3 | HVR-L1 | HVR-L2 | HVR-L3 |
|---|---|---|---|---|---|---|
| TY21 | FTFSDYAI | IGIISPSSGSTNYA | ARDIHSGSSGY | RASESVDFF | DASNR | YCQHYTSS |
| 585 | HWV | QKFQGRV | YYGFDV | GISFLA | ATGI | PPVYT |
| | (SEQ ID | (SEQ ID NO: 30) | (SEQ ID NO: 40) | (SEQ ID NO: | (SEQ ID | (SEQ ID |
| | NO: 18) | | | 53) | NO: 66) | NO: 70) |
| TY21 | YSITSGYY | VSSISGSGSTTYYA | ARDGFGYFDY | SASSSVSYV | DASSLE | YCVQGLQ |
| 586 | WAWI | DSVKGRF | (SEQ ID NO: 41) | Y | SGV | TPWT |
| | (SEQ ID | (SEQ ID NO: 31) | | (SEQ ID NO: | (SEQ ID | (SEQ ID |
| | NO: 19) | | | 54) | NO: 67) | NO: 71) |
| TY21 | FTFSDYGI | IGEIYHSGSTYYSP | ARDVAPGSSG | RASQGIGSSL | DASNR | YCQQYDQ |
| 587 | HWV | SLKSRV | YYDGFDF | A | ATGI | WPPWT |
| | (SEQ ID | (SEQ ID NO: 32) | (SEQ ID NO: 42) | (SEQ ID NO: | (SEQ ID | (SEQ ID |
| | NO: 20) | | | 55) | NO: 66) | NO: 72) |
| TY21 | YSISSGYH | VSGISGYGGSTYY | ARHSYYGSGN | RASESVDFF | DASNL | YCQQSYS |
| 588 | WDWI | ADSVKGRF | FDY | GKSFLH | ETGV | WPPT |
| | (SEQ ID | (SEQ ID NO: 33) | (SEQ ID NO: 43) | (SEQ ID NO: | (SEQ ID | (SEQ ID |
| | NO: 21) | | | 56 | NO: 68) | NO: 73) |
| TY21 | FTFSDYWI | IGWISPSGGGTKY | ARGAYEFDY | RASQSVSSR | DASNR | YCQQSYPT |
| 589 | HWV | AQKFQGRV | (SEQ ID NO: 44) | FLA | ATGI | PLT |
| | (SEQ ID | (SEQ ID NO: 34) | | (SEQ ID NO: | (SEQ ID | (SEQ ID |
| | NO: 22) | | | 57) | NO: 66) | NO: 74) |
| TY21 | YSISSGYH | LARIDWDDDKYY | ARSYVYFDY | RASQSVRGR | DASNR | YCQQSSSW |
| 580 | WSWI | STSLKSRL | (SEQ ID NO: 45) | FLA | ATGI | PPT |
| | (SEQ ID | (SEQ ID NO: 35) | | (SEQ ID NO: | (SEQ ID | (SEQ ID |
| | NO: 23) | | | 58) | NO: 66) | NO: 75) |
| TY21 | FSLSTGGV | IGEIYHSGSTYYSP | ARRIATATYFD | RASQTVFSR | DASNR | YCQQSYY |
| 591 | AVSWI | SLKSRV | Y | YLA | ATGI | WPPWT |
| | (SEQ ID | (SEQ ID NO: 32) | (SEQ ID NO: 46) | (SEQ ID NO: | (SEQ ID | (SEQ ID |
| | NO: 24) | | | 59) | NO: 66) | NO: 76) |
| TY21 | FSLSTGGV | VSAISGYGSTTYY | ARLPYSAYAF | RASQGVSSY | AASTL | YCQHHYG |
| 686 | AVGWI | ADSVKGRF | DY | LA | QSGV | TPLT |
| | (SEQ ID | (SEQ ID NO: 36) | (SEQ ID NO: 47) | (SEQ ID NO: | (SEQ ID | (SEQ ID |
| | NO: 25) | | | 60) | NO: 69) | NO: 77) |
| TY21 | FTFSGYAI | IGIISPSGGGTKYA | ARHPFAY | RASQSVDFY | DASNR | YCQQYVSS |
| 687 | HWV | QKFQGRV | (SEQ ID NO: 48) | GISFLD | ATGI | PPEYT |
| | (SEQ ID | (SEQ ID NO: 37) | | (SEQ ID NO: | (SEQ ID | (SEQ ID |
| | NO: 26) | | | 61) | NO: 66) | NO: 78) |
| TY21 | YTFSGYGI | IGEIYHSGSTYYSP | ARRIDAFDI | RASQSVDFD | DASSLE | YCQQRDS |
| 689 | HWV | SLKSRV | (SEQ ID NO: 49) | GFSFLH | SGV | WPYT |
| | (SEQ ID | (SEQ ID NO: 32) | | (SEQ ID NO: | (SEQ ID | (SEQ ID |
| | NO: 27) | | | 62) | NO: 67) | NO: 79) |
| TY21 | YTFSGYAI | IGIISPSGGGTKYA | ARLYDVAY | RASQSVDFH | DASSLE | YCEQSLEV |
| 680 | HWV | QKFQGRV | (SEQ ID NO: 50) | GKSFLH | SGV | PFT |
| | (SEQ ID | (SEQ ID NO: 37) | | (SEQ ID NO: | (SEQ ID | (SEQ ID |
| | NO: 28) | | | 63) | NO: 67) | NO: 80) |
| TY21 | FTFSDYAI | IGIISPSGGSTKYA | ARLGYGYFDV | RASQSVDFY | DASSLE | YCVQALQ |
| 691 | HWV | QKFQGRV | (SEQ ID NO: 51) | GISFLH | SGV | LPLT |
| | (SEQ ID | (SEQ ID NO: 38) | | (SEQ ID NO: | (SEQ ID | (SEQ ID |
| | NO: 18) | | | 64) | NO: 67) | NO: 81) |
| TY21 | YSITSGHY | IGDISHSGSTYYSQ | ARGSRTGYFD | RASQSISSYL | DASNL | YCQHHYG |
| 692 | WSWI | SLKSRV | Y | N | ETGV | TPLT |
| | (SEQ ID | (SEQ ID NO: 39) | (SEQ ID NO: 52) | (SEQ ID NO: | (SEQ ID | (SEQ ID |
| | NO: 29) | | | 65) | NO: 68) | NO: 77) |

In some embodiments, the antibody comprises an HVR-H1 comprising the amino acid sequence of SEQ ID NO: 18, an HVR-H2 comprising the amino acid sequence of SEQ ID NO: 30, an HVR-H3 comprising the amino acid sequence of SEQ ID NO: 40, an HVR-L1 comprising the amino acid sequence of SEQ ID NO: 53, an HVR-L2 comprising the amino acid sequence of SEQ ID NO: 66, and an HVR-L3 comprising the amino acid sequence of SEQ ID NO: 70. In some embodiments, the antibody comprises an HVR-H1 comprising the amino acid sequence of SEQ ID NO: 19, an HVR-H2 comprising the amino acid sequence of SEQ ID NO: 31, an HVR-H3 comprising the amino acid sequence of SEQ ID NO: 41, an HVR-L1 comprising the amino acid sequence of SEQ ID NO: 54, an HVR-L2 comprising the amino acid sequence of SEQ ID NO: 67, and an HVR-L3 comprising the amino acid sequence of SEQ ID NO: 71. In some embodiments, the antibody comprises an HVR-H1 comprising the amino acid sequence of SEQ ID NO: 20, an HVR-H2 comprising the amino acid sequence of SEQ ID NO: 32, an HVR-H3 comprising the amino acid sequence of SEQ ID NO: 42, an HVR-L1 comprising the amino acid sequence of SEQ ID NO: 55, an HVR-L2 comprising the amino acid sequence of SEQ ID NO: 66, and an HVR-L3 comprising the amino acid sequence of SEQ ID NO: 72. In some embodiments, the antibody comprises an HVR-H1 comprising the amino acid sequence of SEQ ID NO: 21 an HVR-H2 comprising the amino acid sequence of SEQ ID NO: 33, an HVR-H3 comprising the amino acid sequence of SEQ ID NO: 43, an HVR-L1 comprising the amino acid sequence of SEQ ID NO: 56, an HVR-L2 comprising the amino acid sequence of SEQ ID NO: 68, and an HVR-L3 comprising the amino acid sequence of SEQ ID NO: 73. In some embodiments, the antibody comprises an HVR-H1 comprising the amino acid sequence of SEQ ID NO: 22, an HVR-H2 comprising the amino acid sequence of SEQ ID NO: 34, an HVR-H3 comprising the amino acid sequence of SEQ ID NO: 44, an HVR-L1 comprising the amino acid sequence of SEQ ID NO: 57, an HVR-L2 comprising the amino acid sequence of SEQ ID NO: 66, and an HVR-L3 comprising the amino acid sequence of SEQ ID NO: 74. In some embodiments, the antibody comprises an HVR-H1 comprising the amino acid sequence of SEQ ID NO: 23, an HVR-H2 comprising the amino acid sequence of SEQ ID NO: 35, an HVR-H3 comprising the amino acid sequence of SEQ ID NO: 45, an HVR-L1 comprising the amino acid sequence of SEQ ID NO: 58, an HVR-L2 comprising the amino acid sequence of SEQ ID NO: 66, and an HVR-L3 comprising the amino acid sequence of SEQ ID NO: 75. In some embodiments, the antibody comprises an HVR-H1 comprising the amino acid sequence of SEQ ID NO: 24, an HVR-H2 comprising the amino acid sequence of SEQ ID NO: 32, an HVR-H3 comprising the amino acid sequence of SEQ ID NO: 46, an HVR-L1 comprising the amino acid sequence of SEQ ID NO: 59, an HVR-L2 comprising the amino acid sequence of SEQ ID NO: 66, and an HVR-L3 comprising the amino acid sequence of SEQ ID NO: 76. In some embodiments, the antibody comprises an HVR-H1 comprising the amino acid sequence of SEQ ID NO: 25, an HVR-H2 comprising the amino acid sequence of SEQ ID NO: 36, an HVR-H3 comprising the amino acid sequence of SEQ ID NO: 47, an HVR-L1 comprising the amino acid sequence of SEQ ID NO: 60, an HVR-L2 comprising the amino acid sequence of SEQ ID NO: 69, and an HVR-L3 comprising the amino acid sequence of SEQ ID NO: 77. In some embodiments, the antibody comprises an HVR-H1 comprising the amino acid sequence of SEQ ID NO: 26, an HVR-H2 comprising the amino acid sequence of SEQ ID NO: 37, an HVR-H3 comprising the amino acid sequence of SEQ ID NO: 48, an HVR-L1 comprising the amino acid sequence of SEQ ID NO: 61, an HVR-L2 comprising the amino acid sequence of SEQ ID NO: 66, and an HVR-L3 comprising the amino acid sequence of SEQ ID NO: 78. In some embodiments, the antibody comprises an HVR-H1 comprising the amino acid sequence of SEQ ID NO: 27, an HVR-H2 comprising the amino acid sequence of SEQ ID NO: 32, an HVR-H3 comprising the amino acid sequence of SEQ ID NO: 49, an HVR-L1 comprising the amino acid sequence of SEQ ID NO: 62, an HVR-L2 comprising the amino acid sequence of SEQ ID NO: 67, and an HVR-L3 comprising the amino acid sequence of SEQ ID NO: 79. In some embodiments, the antibody comprises an HVR-H1 comprising the amino acid sequence of SEQ ID NO: 28, an HVR-H2 comprising the amino acid sequence of SEQ ID NO: 37, an HVR-H3 comprising the amino acid sequence of SEQ ID NO: 50, an HVR-L1 comprising the amino acid sequence of SEQ ID NO: 63, an HVR-L2 comprising the amino acid sequence of SEQ ID NO: 67, and an HVR-L3 comprising the amino acid sequence of SEQ ID NO: 80. In some embodiments, the antibody comprises an HVR-H1 comprising the amino acid sequence of SEQ ID NO: 18, an HVR-H2 comprising the amino acid sequence of SEQ ID NO: 38, an HVR-H3 comprising the amino acid sequence of SEQ ID NO: 51, an HVR-L1 comprising the amino acid sequence of SEQ ID NO: 64, an HVR-L2 comprising the amino acid sequence of SEQ ID NO: 67, and an HVR-L3 comprising the amino acid sequence of SEQ ID NO: 81. In some embodiments, the antibody comprises an HVR-H1 comprising the amino acid sequence of SEQ ID NO: 29, an HVR-H2 comprising the amino acid sequence of SEQ ID NO: 39, an HVR-H3 comprising the amino acid sequence of SEQ ID NO: 52, an HVR-L1 comprising the amino acid sequence of SEQ ID NO: 65, an HVR-L2 comprising the amino acid sequence of SEQ ID NO: 68, and an HVR-L3 comprising the amino acid sequence of SEQ ID NO: 77.

In some embodiments, the antibody comprises: a) a heavy chain variable region comprising an amino acid sequence selected from SEQ ID NOS: 82-94; and/or b) a light chain variable region comprising an amino acid sequence selected from SEQ ID NOS: 95-107. In some embodiments, the antibody comprises a heavy chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NOS: 82-94, and/or a light chain variable region comprising an amino acid sequence having at least 90% (e.g., at least 90%, 91%, 92%, 93%, 94%, 95%, 96%, 97%, 98%, or 99%) sequence identity to a sequence selected from SEQ ID NOS: 95-107. In some embodiments, the antibody comprises a heavy chain variable region and a light chain variable region of any of the exemplary antibodies described in Table B below. In some embodiments, the antibody comprises one, two, or all three HVRs of the heavy chain variable region, and/or one, two, or all three HVRs of the light chain variable region shown for any of the exemplary antibodies described in Table B below.

TABLE B
anti-CTLA4 variable region amino acid sequences

| Ab name: | VH: | VL: |
|---|---|---|
| TY21585 | EVQLVESGGGLVQPGGSLRLSCAASGFTFSDY | DIQLTQSPSSLSASVGDRVTITCRASESVDFF |
| | AIHWVRQAPGKGLEWIGIISPSSGSTNYAQKF | GISFLAWYQQKPGKAPKLLIYDASNRATGI |
| | QGRVTISRDNSKNTLYLQLNSLRAEDTAVYYC | PSRFSGSGSGTDFTLTISSLQPEDFATYYCQ |
| | ARDIHSGSSGYYYGFDVWGQGTLVTVSS | HYTSSPPVYTFGQGTKVEIKR |
| | (SEQ ID NO: 82) | (SEQ ID NO: 95) |
| TY21586 | EVQLVESGGGLVQPGGSLRLSCAASGYSITSG | DIQLTQSPSSLSASVGDRVTITCSASSSVSYV |
| | YYWAWIRQAPGKGLEWVSSISGSGSTTYYAD | YWYQQKPGKAPKLLIYDASSLESGVPSRFS |
| | SVKGRFTISRDNSKNTLYLQLNSLRAEDTAVY | GSGSGTDFTLTISSLQPEDFATYYCVQGLQT |
| | YCARDGFGYFDYWGQGTLVTVSS | PWTFGQGTKVEIKR |
| | (SEQ ID NO: 83) | (SEQ ID NO: 96) |
| TY21587 | EVQLVESGGGLVQPGGSLRLSCAASGFTFSDY | DIQLTQSPSSLSASVGDRVTITCRASQGIGSS |
| | GIHWVRQAPGKGLEWIGEIYHSGSTYYSPSLK | LAWYQQKPGKAPKLLIYDASNRATGIPSRF |
| | SRVTISRDNSKNTLYLQLNSLRAEDTAVYYCA | SGSGSGTDFTLTISSLQPEDFATYYCQQYDQ |
| | RDVAPGSSGYYDGFDFWGQGTLVTVSS | WPPWTFGQGTKVEIKR |
| | (SEQ ID NO: 84) | (SEQ ID NO: 97) |
| TY21588 | EVQLVESGGGLVQPGGSLRLSCAASGYSISSG | DIQLTQSPSSLSASVGDRVTITCRASESVDFF |
| | YHWDWIRQAPGKGLEWVSGISGYGGSTYYA | GKSFLHWYQQKPGKAPKLLIYDASNLETG |
| | DSVKGRFTISRDNSKNTLYLQLNSLRAEDTAV | VPSRFSGSGSGTDFTLTISSLQPEDFATYYC |
| | YYCARHSYYGSGNFDYWGQGTLVTVSS | QQSYSWPPTFGQGTKVEIKR |
| | (SEQ ID NO: 85) | (SEQ ID NO: 98) |
| TY21589 | EVQLVESGGGLVQPGGSLRLSCAASGFTFSDY | DIQLTQSPSSLSASVGDRVTITCRASQSVSS |
| | WIHWVRQAPGKGLEWIGWISPSGGGTKYAQK | RFLAWYQQKPGKAPKLLIYDASNRATGIPS |
| | FQGRVTISRDNSKNTLYLQLNSLRAEDTAVYY | RFSGSGSGTDFTLTISSLQPEDFATYYCQQS |
| | CARGAYEFDYWGQGTLVTVSS | YPTPLTFGQGTKVEIKR |
| | (SEQ ID NO: 86) | (SEQ ID NO: 99) |
| TY21580 | EVQLVESGGGLVQPGGSLRLSCAASGYSISSG | DIQLTQSPSSLSASVGDRVTITCRASQSVRG |
| | YHWSWIRQAPGKGLEWLARIDWDDDKYYST | RFLAWYQQKPGKAPKLLIYDASNRATGIPS |
| | SLKSRLTISRDNSKNTLYLQLNSLRAEDTAVYY | RFSGSGSGTDFTLTISSLQPEDFATYYCQQS |
| | CARSYVYFDYWGQGTLVTVSS | SSWPPTFGQGTKVEIKR |
| | (SEQ ID NO: 87) | (SEQ ID NO: 100) |
| TY21591 | EVQLVESGGGLVQPGGSLRLSCAASGFSLSTG | DIQLTQSPSSLSASVGDRVTITCRASQTVFS |
| | GVAVSWIRQAPGKGLEWIGEIYHSGSTYYSPS | RYLAWYQQKPGKAPKLLIYDASNRATGIPS |
| | LKSRVTISRDNSKNTLYLQLNSLRAEDTAVYY | RFSGSGSGTDFTLTISSLQPEDFATYYCQQS |
| | CARRIATATYFDYWGQGTLVTVSS | YYWPPWTFGQGTKVEIKR |
| | (SEQ ID NO: 88) | (SEQ ID NO: 101) |
| TY21686 | EVQLVESGGGLVQPGGSLRLSCAASGFSLSTG | DIQLTQSPSSLSASVGDRVTITCRASQGVSS |
| | GVAVGWIRQAPGKGLEWVSAISGYGSTTYYA | YLAWYQQKPGKAPKLLIYAASTLQSGVPSR |
| | DSVKGRFTISRDNSKNTLYLQLNSLRAEDTAV | FSGSGSGTDFTLTISSLQPEDFATYYCQHHY |
| | YYCARLPYSAYAFDYWGQGTLVTVSS | GTPLTFGQGTKVEIKR |
| | (SEQ ID NO: 89) | (SEQ ID NO: 102) |
| TY21687 | EVQLVESGGGLVQPGGSLRLSCAASGFTFSGY | DIQLTQSPSSLSASVGDRVTITCRASQSVDF |
| | AIHWVRQAPGKGLEWIGIISPSGGGTKYAQKF | YGISFLDWYQQKPGKAPKLLIYDASNRATG |
| | QGRVTISRDNSKNTLYLQLNSLRAEDTAVYYC | IPSRFSGSGSGTDFTLTISSLQPEDFATYYCQ |
| | ARHPFAYWGQGTLVTVSS | QYVSSPPEYTFGQGTKVEIKR |
| | (SEQ ID NO: 90) | (SEQ ID NO: 103) |
| TY21689 | EVQLVESGGGLVQPGGSLRLSCAASGYTFSGY | DIQLTQSPSSLSASVGDRVTITCRASQSVDF |
| | GIHWVRQAPGKGLEWIGEIYHSGSTYYSPSLK | DGFSFLHWYQQKPGKAPKLLIYDASSLESG |
| | SRVTISRDNSKNTLYLQLNSLRAEDTAVYYCA | VPSRFSGSGSGTDFTLTISSLQPEDFATYYC |
| | RRIDAFDIWGQGTLVTVSS | QQRDSWPYTFGQGTKVEIKR |
| | (SEQ ID NO: 91) | (SEQ ID NO: 104) |
| TY21680 | EVQLVESGGGLVQPGGSLRLSCAASGYTFSGY | DIQLTQSPSSLSASVGDRVTITCRASQSVDF |
| | AIHWVRQAPGKGLEWIGIISPSGGGTKYAQKF | HGKSFLHWYQQKPGKAPKLLIYDASSLES |
| | QGRVTISRDNSKNTLYLQLNSLRAEDTAVYYC | GVPSRFSGSGSGTDFTLTISSLQPEDFATYY |
| | ARLYDVAYWGQGTLVTVSS | CEQSLEVPFTFGQGTK VEIKR |
| | (SEQ ID NO: 92) | (SEQ ID NO: 105) |
| TY21691 | EVQLVESGGGLVQPGGSLRLSCAASGFTFSDY | DIQLTQSPSSLSASVGDRVTITCRASQSVDF |
| | AIHWVRQAPGKGLEWIGIISPSGGSTKYAQKF | YGISFLHWYQQKPGKAPKLLIYDASSLESG |
| | QGRVTISRDNSKNTLYLQLNSLRAEDTAVYYC | VPSRFSGSGSGTDFTLTISSLQPEDFATYYC |
| | ARLGYGYFDVWGQGTLVTVSS | VQALQLPLTFGQGTKVEIKR |
| | (SEQ ID NO: 93) | (SEQ ID NO: 106) |
| TY21692 | EVQLVESGGGLVQPGGSLRLSCAASGYSITSG | DIQLTQSPSSLSASVGDRVTITCRASQSISSY |
| | HYWSWIRQAPGKGLEWIGDISHSGSTYYSQSL | LNWYQQKPGKAPKLLIYDASNLETGVPSR |
| | KSRVTISRDNSKNTLYLQLNSLRAEDTAVYYC | FSGSGSGTDFTLTISSLQPEDFATYYCQHHY |
| | ARGSRTGYFDYWGQGTLVTVSS | GTPLTFGQGTKVEIKR |
| | (SEQ ID NO: 94) | (SEQ ID NO: 107) |

In some embodiments, the antibody comprises a heavy chain variable region comprising the amino acid sequence of SEQ ID NO: 82, and a light chain variable region comprising the amino acid sequence of SEQ ID NO: 95. In some embodiments, the antibody comprises a heavy chain variable region comprising the amino acid sequence of SEQ ID NO: 83, and a light chain variable region comprising the amino acid sequence of SEQ ID NO: 96. In some embodiments, the antibody comprises a heavy chain variable region comprising the amino acid sequence of SEQ ID NO: 84, and a light chain variable region comprising the amino acid sequence of SEQ ID NO: 97. In some embodiments, the antibody comprises a heavy chain variable region comprising the amino acid sequence of SEQ ID NO: 85, and a light chain variable region comprising the amino acid sequence of SEQ ID NO: 98. In some embodiments, the antibody comprises a heavy chain variable region comprising the amino acid sequence of SEQ ID NO: 86, and a light chain variable region comprising the amino acid sequence of SEQ ID NO: 99. In some embodiments, the antibody comprises a heavy chain variable region comprising the amino acid sequence of SEQ ID NO: 87, and a light chain variable region comprising the amino acid sequence of SEQ ID NO: 100. In some embodiments, the antibody comprises a heavy chain variable region comprising the amino acid sequence of SEQ ID NO: 88, and a light chain variable region comprising the amino acid sequence of SEQ ID NO: 101. In some embodiments, the antibody comprises a heavy chain variable region comprising the amino acid sequence of SEQ ID NO: 89, and a light chain variable region comprising the amino acid sequence of SEQ ID NO: 102. In some embodiments, the antibody comprises a heavy chain variable region comprising the amino acid sequence of SEQ ID NO: 90, and a light chain variable region comprising the amino acid sequence of SEQ ID NO: 103. In some embodiments, the antibody comprises a heavy chain variable region comprising the amino acid sequence of SEQ ID NO: 91, and a light chain variable region comprising the amino acid sequence of SEQ ID NO: 104. In some embodiments, the antibody comprises a heavy chain variable region comprising the amino acid sequence of SEQ ID NO: 92, and a light chain variable region comprising the amino acid sequence of SEQ ID NO: 105. In some embodiments, the antibody comprises a heavy chain variable region comprising the amino acid sequence of SEQ ID NO: 93, and a light chain variable region comprising the amino acid sequence of SEQ ID NO: 106. In some embodiments, the antibody comprises a heavy chain variable region comprising the amino acid sequence of SEQ ID NO: 94, and a light chain variable region comprising the amino acid sequence of SEQ ID NO: 107.

In some embodiments, an antibody of the present application cross-competes for binding to human CTLA4 with an antibody comprising: a) an HVR-H1 comprising an amino acid sequence selected from SEQ ID NOS: 18-29; an HVR-H2 comprising an amino acid sequence selected from SEQ ID NOS: 30-39; and an HVR-H3 comprising an amino acid sequence selected from SEQ ID NOS: 40-52; and/or b) an HVR-L1 comprising an amino acid sequence selected from SEQ ID NOS: 53-65; an HVR-L2 comprising an amino acid sequence selected from SEQ ID NOS: 66-69; and an HVR-L3 comprising an amino acid sequence selected from SEQ ID NOS: 70-81. In some embodiments, an antibody of the present application cross-competes for binding to human CTLA4 with an antibody comprising one, two, three, four, five, or all six of the HVRs shown for any of the exemplary antibodies described in Table A. In some embodiments, an antibody of the present application cross-competes for binding to human CTLA4 with an antibody comprising: a) a heavy chain variable region comprising an amino acid sequence selected from SEQ ID NOS: 82-94; and/or b) a light chain variable region comprising an amino acid sequence selected from SEQ ID NOS: 95-107. In some embodiments, an antibody of the present application cross-competes for binding to human CTLA4 with an antibody comprising a VH and/or VL shown for any of the exemplary antibodies described in Table B.

The CTLA4 antibodies described herein may be in any class, such as IgG, IgM, IgE, IgA, or IgD. In some embodiments, the CTLA4 antibodies are in the IgG class, such as IgG1, IgG2, IgG3, or IgG4 subclass. A CTLA4 antibody can be converted from one class or subclass to another class or subclass using methods known in the art. An exemplary method for producing an antibody in a desired class or subclass comprises the steps of isolating a nucleic acid encoding a heavy chain of a CTLA4 antibody and a nucleic acid encoding a light chain of a CTLA4 antibody, isolating the sequence encoding the V_{H} region, ligating the V_{H} sequence to a sequence encoding a heavy chain constant region of the desired class or subclass, expressing the light chain gene and the heavy chain construct in a cell, and collecting the CTLA4 antibody. Antibodies of the present application may be monoclonal antibodies or polyclonal antibodies. Antibodies of the present application may be monospecific antibodies or multispecific (e.g., bispecific, trispecific, etc.) antibodies. In some embodiments, the CTLA4 antibodies described herein may include one or more Fc mutations (e.g., that modulate (increase or decrease) ADCC or CDC activities). Any suitable Fc mutations known in the art may be used in the CTLA4 antibodies of the present application.

In some embodiments, the anti-CTLA4 antibody comprises a heavy chain comprising the amino acid sequence EVQLVESGGGLVQPGGSLRLSCAASGYSISSGYHWSWIRQAPGKGLEWLARIDWDDDKYYSTSL KSRLTISRDNSKNTLYLQLNSLRAEDTAVYYCARSYVYFDYWGQGTLVTVSSASTKGPSVFPLA PSSKSTSGGTAALGCLVKDYFPEPVTVSWNSGALTSGVHTFPAVLQSSGLYSLSSVVTVPSSSL GTQTYICNVNHKPSNTKVDKKVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTQKSLSLSPG (SEQ ID NO:125) and a light chain comprising the amino acid sequence DIQLTQSPSSLSASVGDRVTITCRASQSVRGRFLAWYQQKPGKAPKLLIYDASNRATGIPSRFS GSGSGTDFTLTISSLQPEDFATYYCQQSSSWPPTFGQGTKVEIKRTVAAPSVFIFPPSDEQLKS GTASVVCLLNNFYPREAKVQWKVDNALQSGNSQESVTEQDSKDSTYSLSSTLTLSKADYEKHKV YACEVTHQGLSSPVTKSFNRGEC (SEQ ID NO:127). In some embodiments, the anti-CTLA4 antibody comprises a heavy chain comprising the amino acid sequence EVQLVESGGGLVQPGGSLRLSCAASGYSISSGYHWSWIRQAPGKGLEWLARIDWDDDKYYSTSL KSRLTISRDNSKNTLYLQLNSLRAEDTAVYYCARSYVYFDYWGQGTLVTVSSASTKGPSVFPLA PSSKSTSGGTAALGCLVKDYFPEPVTVSWNSGALTSGVHTFPAVLQSSGLYSLSSVVTVPSSSL GTQTYICNVNHKPSNTKVDKKVEPKSCDKTHTCPPCPAPELLGGPSVFLFPPKPKDTLMISRTP EVTCVVVDVSHEDPEVKFNWYVDGVEVHNAKTKPREEQYNSTYRVVSVLTVLHQDWLNGKEYKC KVSNKALPAPIEKTISKAKGQPREPQVYTLPPSRDELTKNQVSLTCLVKGFYPSDIAVEWESNG QPENNYKTTPPVLDSDGSFFLYSKLTVDKSRWQQGNVFSCSVMHEALHNHYTQKSLSLSPGK (SEQ ID NO:126) and a light chain comprising the amino acid sequence DIQLTQSPSSLSASVGDRVTITCRASQSVRGRFLAWYQQKPGKAPKLLIYDASNRATGIPSRFS GSGSGTDFTLTISSLQPEDFATYYCQQSSSWPPTFGQGTKVEIKRTVAAPSVFIFPPSDEQLKS GTASVVCLLNNFYPREAKVQWKVDNALQSGNSQESVTEQDSKDSTYSLSSTLTLSKADYEKHKV YACEVTHQGLSSPVTKSFNRGEC (SEQ ID NO:127). In some embodiments, the anti-CTLA4 antibody refers to a mix of antibody species, wherein each antibody species has a light chain comprising the amino acid sequence of SEQ ID NO: 127 and a heavy chain comprising either the amino acid sequence of SEQ ID NO: 125 or 126.

In some embodiments, the anti-CTLA4 antibody comprises a heavy chain comprising the amino acid sequence EVQLVESGGGLVQPGGSLRLSCAASGFTFSDYAIHWVRQAPGKGLEWIGIISPSGGSTKYAQKF QGRVTISRDNSKNTLYLQLNSLRAEDTAVYYCARLGYGYFDVWGQGTLVTVSSASTKGPSVFPL APSSKSTSGGTAALGCLVKDYFPEPVTVSWNSGALTSGVHTFPAVLQSSGLYSLSSVVTVPSSS LGTQTYICNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFYPSDIAVEWESN GQPENNYKTTPPVLDSDGSFFLYSKLTVDKSRWQQGNVFSCSVMHEALHNHYTQKSLSLSPG (SEQ ID NO:128) and a light chain comprising the amino acid sequence DIQLTQSPSSLSASVGDRVTITCRASQSVDFYGISFLHWYQQKPGKAPKLLIYDASSLESGVPS RFSGSGSGTDFTLTISSLQPEDFATYYCVQALQLPLTFGQGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:130). In some embodiments, the anti-CTLA4 antibody comprises a heavy chain comprising the amino acid sequence EVQLVESGGGLVQPGGSLRLSCAASGFTFSDYAIHWVRQAPGKGLEWIGIISPSGGSTKYAQKF QGRVTISRDNSKNTLYLQLNSLRAEDTAVYYCARLGYGYFDVWGQGTLVTVSSASTKGPSVFPL APSSKSTSGGTAALGCLVKDYFPEPVTVSWNSGALTSGVHTFPAVLQSSGLYSLSSVVTVPSSS LGTQTYICNVNHKPSNTKVDKKVEPKSCDKTHTCPPCPAPELLGGPSVFLFPPKPKDTLMISRT PEVTCVVVDVSHEDPEVKFNWYVDGVEVHNAKTKPREEQYNSTYRVVSVLTVLHQDWLNGKEYK CKVSNKALPAPIEKTISKAKGQPREPQVYTLPPSRDELTKNQVSLTCLVKGFYPSDIAVEWESN GQPENNYKTTPPVLDSDGSFFLYSKLTVDKSRWQQGNVFSCSVMHEALHNHYTQKSLSLSPGK (SEQ ID NO:129) and a light chain comprising the amino acid sequence DIQLTQSPSSLSASVGDRVTITCRASQSVDFYGISFLHWYQQKPGKAPKLLIYDASSLESGVPS RFSGSGSGTDFTLTISSLQPEDFATYYCVQALQLPLTFGQGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:130). In some embodiments, the anti-CTLA4 antibody refers to a mix of antibody species, wherein each antibody species has a light chain comprising the amino acid sequence of SEQ ID NO: 130 and a heavy chain comprising either the amino acid sequence of SEQ ID NO: 128 or 129.

In some embodiments, the anti-CTLA4 antibody comprises a heavy chain comprising the amino acid sequence EVQLVESGGGLVQPGGSLRLSCAASGYSITSGYYWAWIRQAPGKGLEWVSSISGSGSTTYYADS VKGRFTISRDNSKNTLYLQLNSLRAEDTAVYYCARDGFGYFDYWGQGTLVTVSSASTKGPSVFP LAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLNGKEY KCKVSNKALPAPIEKTISKAKGQPREPQVYTLPPSRDELTKNQVSLTCLVKGFYPSDIAVEWES NGQPENNYKTTPPVLDSDGSFFLYSKLTVDKSRWQQGNVFSCSVMHEALHNHYTQKSLSLSPG (SEQ ID NO:131) and a light chain comprising the amino acid sequence DIQLTQSPSSLSASVGDRVTITCSASSSVSYVYWYQQKPGKAPKLLIYDASSLESGVPSRFSGS GSGTDFTLTISSLQPEDFATYYCVQGLQTPWTFGQGTKVEIKRTVAAPSVFIFPPSDEQLKSGT ASVVCLLNNFYPREAKVQWKVDNALQSGNSQESVTEQDSKDSTYSLSSTLTLSKADYEKHKVYA CEVTHQGLSSPVTKSFNRGEC (SEQ ID NO:133). In some embodiments, the anti-CTLA4 antibody comprises a heavy chain comprising the amino acid sequence EVQLVESGGGLVQPGGSLRLSCAASGYSITSGYYWAWIRQAPGKGLEWVSSISGSGSTTYYADS VKGRFTISRDNSKNTLYLQLNSLRAEDTAVYYCARDGFGYFDYWGQGTLVTVSSASTKGPSVFP LAPSSKSTSGGTAALGCLVKDYFPEPVTVSWNSGALTSGVHTFPAVLQSSGLYSLSSVVTVPSS SLGTQTYICNVNHKPSNTKVDKKVEPKSCDKTHTCPPCPAPELLGGPSVFLFPPKPKDTLMISR TPEVTCVVVDVSHEDPEVKFNWYVDGVEVHNAKTKPREEQYNSTYRVVSVLTVLHQDWLNGKEY KCKVSNKALPAPIEKTISKAKGQPREPQVYTLPPSRDELTKNQVSLTCLVKGFYPSDIAVEWES NGQPENNYKTTPPVLDSDGSFFLYSKLTVDKSRWQQGNVFSCSVMHEALHNHYTQKSLSLSPGK (SEQ ID NO:132) and a light chain comprising the amino acid sequence DIQLTQSPSSLSASVGDRVTITCSASSSVSYVYWYQQKPGKAPKLLIYDASSLESGVPSRFSGS GSGTDFTLTISSLQPEDFATYYCVQGLQTPWTFGQGTKVEIKRTVAAPSVFIFPPSDEQLKSGT ASVVCLLNNFYPREAKVQWKVDNALQSGNSQESVTEQDSKDSTYSLSSTLTLSKADYEKHKVYA CEVTHQGLSSPVTKSFNRGEC (SEQ ID NO:133). In some embodiments, the anti-CTLA4 antibody refers to a mix of antibody species, wherein each antibody species has a light chain comprising the amino acid sequence of SEQ ID NO: 133 and a heavy chain comprising either the amino acid sequence of SEQ ID NO: 131 or 132.

In some embodiments, the anti-CTLA4 antibody comprises a heavy chain comprising the amino acid sequence EVQLVESGGGLVQPGGSLRLSCAASGFTFSGYAIHWVRQAPGKGLEWIGIISPSGGGTKYAQKF QGRVTISRDNSKNTLYLQLNSLRAEDTAVYYCARHPFAYWGQGTLVTVSSASTKGPSVFPLAPS SKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEV TCVVVDVSHEDPEVKFNWYVDGVEVHNAKTKPREEQYNSTYRVVSVLTVLHQDWLNGKEYKCKV SNKALPAPIEKTISKAKGQPREPQVYTLPPSRDELTKNQVSLTCLVKGFYPSDIAVEWESNGQP ENNYKTTPPVLDSDGSFFLYSKLTVDKSRWQQGNVFSCSVMHEALHNHYTQKSLSLSPG (SEQ ID NO:134) and a light chain comprising the amino acid sequence DIQLTQSPSSLSASVGDRVTITCRASQSVDFYGISFLDWYQQKPGKAPKLLIYDASNRATGIPS RFSGSGSGTDFTLTISSLQPEDFATYYCQQYVSSPPEYTFGQGTKVEIKRTVAAPSVFIFPPSD EQLKSGTASVVCLLNNFYPREAKVQWKVDNALQSGNSQESVTEQDSKDSTYSLSSTLTLSKADY EKHKVYACEVTHQGLSSPVTKSFNRGEC (SEQ ID NO:136). In some embodiments, the anti-CTLA4 antibody comprises a heavy chain comprising the amino acid sequence EVQLVESGGGLVQPGGSLRLSCAASGFTFSGYAIHWVRQAPGKGLEWIGIISPSGGGTKYAQKF QGRVTISRDNSKNTLYLQLNSLRAEDTAVYYCARHPFAYWGQGTLVTVSSASTKGPSVFPLAPS SKSTSGGTAALGCLVKDYFPEPVTVSWNSGALTSGVHTFPAVLQSSGLYSLSSVVTVPSSSLGT QTYICNVNHKPSNTKVDKKVEPKSCDKTHTCPPCPAPELLGGPSVFLFPPKPKDTLMISRTPEV TCVVVDVSHEDPEVKFNWYVDGVEVHNAKTKPREEQYNSTYRVVSVLTVLHQDWLNGKEYKCKV SNKALPAPIEKTISKAKGQPREPQVYTLPPSRDELTKNQVSLTCLVKGFYPSDIAVEWESNGQP ENNYKTTPPVLDSDGSFFLYSKLTVDKSRWQQGNVFSCSVMHEALHNHYTQKSLSLSPGK (SEQ ID NO:135) and a light chain comprising the amino acid sequence DIQLTQSPSSLSASVGDRVTITCRASQSVDFYGISFLDWYQQKPGKAPKLLIYDASNRATGIPS RFSGSGSGTDFTLTISSLQPEDFATYYCQQYVSSPPEYTFGQGTKVEIKRTVAAPSVFIFPPSD EQLKSGTASVVCLLNNFYPREAKVQWKVDNALQSGNSQESVTEQDSKDSTYSLSSTLTLSKADY EKHKVYACEVTHQGLSSPVTKSFNRGEC (SEQ ID NO:136). In some embodiments, the anti-CTLA4 antibody refers to a mix of antibody species, wherein each antibody species has a light chain comprising the amino acid sequence of SEQ ID NO: 136 and a heavy chain comprising either the amino acid sequence of SEQ ID NO: 134 or 135.

In some embodiments, the anti-CTLA4 antibody comprises a heavy chain comprising the amino acid sequence EVQLVESGGGLVQPGGSLRLSCAASGYTFSGYAIHWVRQAPGKGLEWIGIISPSGGGTKYAQKF QGRVTISRDNSKNTLYLQLNSLRAEDTAVYYCARLYDVAYWGQGTLVTVSSASTKGPSVFPLAP SSKSTSGGTAALGCLVKDYFPEPVTVSWNSGALTSGVHTFPAVLQSSGLYSLSSVVTVPSSSLG TQTYICNVNHKPSNTKVDKKVEPKSCDKTHTCPPCPAPELLGGPSVFLFPPKPKDTLMISRTPE VTCVVVDVSHEDPEVKFNWYVDGVEVHNAKTKPREEQYNSTYRVVSVLTVLHQDWLNGKEYKCK VSNKALPAPIEKTISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYTQKSLSLSPG (SEQ ID NO:137) and a light chain comprising the amino acid sequence DIQLTQSPSSLSASVGDRVTITCRASQSVDFHGKSFLHWYQQKPGKAPKLLIYDASSLESGVPS RFSGSGSGTDFTLTISSLQPEDFATYYCEQSLEVPFTFGQGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:139). In some embodiments, the anti-CTLA4 antibody comprises a heavy chain comprising the amino acid sequence EVQLVESGGGLVQPGGSLRLSCAASGYTFSGYAIHWVRQAPGKGLEWIGIISPSGGGTKYAQKF QGRVTISRDNSKNTLYLQLNSLRAEDTAVYYCARLYDVAYWGQGTLVTVSSASTKGPSVFPLAP SSKSTSGGTAALGCLVKDYFPEPVTVSWNSGALTSGVHTFPAVLQSSGLYSLSSVVTVPSSSLG TQTYICNVNHKPSNTKVDKKVEPKSCDKTHTCPPCPAPELLGGPSVFLFPPKPKDTLMISRTPE VTCVVVDVSHEDPEVKFNWYVDGVEVHNAKTKPREEQYNSTYRVVSVLTVLHQDWLNGKEYKCK VSNKALPAPIEKTISKAKGQPREPQVYTLPPSRDELTKNQVSLTCLVKGFYPSDIAVEWESNGQ PENNYKTTPPVLDSDGSFFLYSKLTVDKSRWQQGNVFSCSVMHEALHNHYTQKSLSLSPGK (SEQ ID NO:138) and a light chain comprising the amino acid sequence DIQLTQSPSSLSASVGDRVTITCRASQSVDFHGKSFLHWYQQKPGKAPKLLIYDASSLESGVPS RFSGSGSGTDFTLTISSLQPEDFATYYCEQSLEVPFTFGQGTKVEIKRTVAAPSVFIFPPSDEQ LKSGTASVVCLLNNFYPREAKVQWKVDNALQSGNSQESVTEQDSKDSTYSLSSTLTLSKADYEK HKVYACEVTHQGLSSPVTKSFNRGEC (SEQ ID NO:139). In some embodiments, the anti-CTLA4 antibody refers to a mix of antibody species, wherein each antibody species has a light chain comprising the amino acid sequence of SEQ ID NO: 139 and a heavy chain comprising either the amino acid sequence of SEQ ID NO: 137 or 138.

In some embodiments, the anti-CTLA4 antibody is an antigen-binding fragment of an anti-CTLA4 antibody. the antigen-binding fragments of a CTLA4 antibody include: (i) a Fab fragment, which is a monovalent fragment consisting of the V_{L}, V_{H}, C_{L} and C_{H}1 domains; (ii) a F(ab′)₂ fragment, which is a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody; (v) a dAb fragment (Ward et al., (1989) Nature 341:544-546), which consists of a V_{H} domain; (vi) an isolated CDR, and (vii) single chain antibody (scFv), which is a polypeptide comprising a V_{L} region of an antibody linked to a V_{H}region of an antibody (see e.g., Bird et al. (1988) Science 242:423-426; Huston et al. (1988) Proc. Natl. Acad. Sci. USA 85:5879-5883).

In some embodiments, the anti-CTLA4 antibody is a derivative of any one of the anti-CTLA4 antibodies described herein. In some embodiments, the antibody derivative is derived from modifications of the amino acid sequences of an illustrative antibody (e.g., a “parent antibody”) of the present application while conserving the overall molecular structure of the parent antibody amino acid sequence. Amino acid sequences of any regions of the parent antibody chains may be modified, such as framework regions, HVR regions, or constant regions. Types of modifications include substitutions, insertions, deletions, or combinations thereof, of one or more amino acids of the parent antibody.

In some embodiments, the antibody derivative comprises a V_{L} or V_{H}region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 82-107 In some embodiments, the antibody derivative comprises an HVR-H1 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 18-29. In some embodiments, the antibody derivative comprises an HVR-H2 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 30-39. In some embodiments, the antibody derivative comprises an HVR-H3 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 40-52. In some embodiments, the antibody derivative comprises an HVR-L1 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 53-65. In some embodiments, the antibody derivative comprises an HVR-L2 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 66-69. In some embodiments, the antibody derivative comprises an HVR-L3 amino acid sequence region that is at least 65%, at least 75%, at least 85%, at least 90%, at least 91%, at least 92%, at least 93%, at least 94%, at least 95%, at least 96%, at least 97%, at least 98%, or at least 99% identical to an amino acid sequence as set forth in any of SEQ ID NOS: 70-81.

In some particular embodiments, the derivative comprises 1, 2, 3, 4, 5, 6, 7, 8, 9, 10, 11, 12, 13, 14, or 15 conservative or non-conservative substitutions, and/or 1, 2, 3, 4, 5, 6, 7, 8, 9, 10, 11, 12, 13, 14, or 15 additions and/or deletions to an amino acid sequence as set forth in any of SEQ ID NOS: 18-107.

Amino acid substitutions encompass both conservative substitutions and non-conservative substitutions. The term “conservative amino acid substitution” means a replacement of one amino acid with another amino acid where the two amino acids have similarity in certain physico-chemical properties such as polarity, charge, solubility, hydrophobicity, hydrophilicity, and/or the amphipathic nature of the residues involved. For example, substitutions typically may be made within each of the following groups: (a) nonpolar (hydrophobic) amino acids, such as alanine, leucine, isoleucine, valine, proline, phenylalanine, tryptophan, and methionine; (b) polar neutral amino acids, such as glycine, serine, threonine, cysteine, tyrosine, asparagine, and glutamine; (c) positively charged (basic) amino acids, such as arginine, lysine, and histidine; and (d) negatively charged (acidic) amino acids, such as aspartic acid and glutamic acid.

The modifications may be made in any positions of the amino acid sequences of the antibody, including the HVRs, framework regions, or constant regions. In one embodiment, the present application provides an antibody derivative that contains the V_{H} and V_{L} HVR sequences of an illustrative antibody of this disclosure, yet contains framework sequences different from those of the illustrative antibody. Such framework sequences can be obtained from public DNA databases or published references that include germline antibody gene sequences. For example, germline DNA sequences for human heavy and light chain variable region genes can be found in the Genbank database or in the “VBase” human germline sequence database (Kaba et al., Sequences of Proteins of Immunological Interest, Fifth Edition, U.S. Department of Health and Human Services, NIH Publication No. 91-3242 (1991); Tomlinson et al., J. Mol. Biol. 227:776-798 (1992); and Cox et al., Eur. J. Immunol. 24:827-836 (1994)). Framework sequences that may be used in constructing an antibody derivative include those that are structurally similar to the framework sequences used by illustrative antibodies of the disclosure For example, the HVR-H1, HVR-H2, and HVR-H3 sequences, and the HVR-L1, HVR-L2, and HVR-L3 sequences of an illustrative antibody can be grafted onto framework regions that have the identical sequence as that found in the germline immunoglobulin gene from which the framework sequence derive, or the HVR sequences can be grafted onto framework regions that contain one or more mutations as compared to the germline sequences.

In some embodiments, the antibody derivative is a chimeric antibody, which comprises an amino acid sequence of an illustrative antibody of the disclosure. In one example, one or more HVRs from one or more illustrative antibodies are combined with HVRs from an antibody from a non-human animal, such as mouse or rat. In another example, all of the HVRs of the chimeric antibody are derived from one or more illustrative antibodies. In some particular embodiments, the chimeric antibody comprises one, two, or three HVRs from the heavy chain variable region and/or one, two, or three HVRs from the light chain variable region of an illustrative antibody. Chimeric antibodies can be generated using conventional methods known in the art.

Another type of modification is to mutate amino acid residues within the HVR regions of the V_{H} and/or V_{L} chain. Site-directed mutagenesis or PCR-mediated mutagenesis can be performed to introduce the mutation(s) and the effect on antibody binding, or other functional property of interest, can be evaluated in in vitro or in vivo assays known in the art. Typically, conservative substitutions are introduced. The mutations may be amino acid additions and/or deletions. Moreover, typically no more than one, two, three, four or five residues within an HVR region are altered. In some embodiments, the antibody derivative comprises 1, 2, 3, or 4 amino acid substitutions in the heavy chain HVRs and/or in the light chain HVRs. In another embodiment, the amino acid substitution is to change one or more cysteines in an antibody to another residue, such as, without limitation, alanine or serine. The cysteine may be a canonical or non-canonical cysteine. In one embodiment, the antibody derivative has 1, 2, 3, or 4 conservative amino acid substitutions in the heavy chain HVR regions relative to the amino acid sequences of an illustrative antibody.

Modifications may also be made to the framework residues within the V_{H} and/or V_{L}regions. Typically, such framework variants are made to decrease the immunogenicity of the antibody. One approach is to “back mutate” one or more framework residues to the corresponding germline sequence. An antibody that has undergone somatic mutation may contain framework residues that differ from the germline sequence from which the antibody is derived. Such residues can be identified by comparing the antibody framework sequences to the germline sequences from which the antibody is derived. To return the framework region sequences to their germline configuration, the somatic mutations can be “back mutated” to the germline sequence by, for example, site-directed mutagenesis or PCR-mediated mutagenesis.

In addition, modifications may also be made within the Fc region of an illustrative antibody, typically to alter one or more functional properties of the antibody, such as serum half-life, complement fixation, Fc receptor binding, and/or antigen-dependent cellular cytotoxicity. In one example, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody. In another case, the Fc hinge region of an antibody is mutated to decrease the biological half-life of the antibody.

Furthermore, an antibody of the present application may be modified to alter its potential glycosylation site or pattern in accordance with routine experimentation known in the art. In another aspect, the present application provides a derivative of a CTLA4 antibody that contains at least one mutation in a variable region of a light chain or heavy chain that changes the pattern of glycosylation in the variable region. Such an antibody derivative may have an increased affinity and/or a modified specificity for binding an antigen. The mutations may add a novel glycosylation site in the V region, change the location of one or more V region glycosylation site(s), or remove a pre-existing V region glycosylation site. In one embodiment, the present application provides a derivative of a CTLA4 antibody having a potential N-linked glycosylation site at asparagine in the heavy chain variable region, wherein the potential N-linked glycosylation site in one heavy chain variable region is removed. In another embodiment, the present application provides a derivative of a CTLA4 antibody having a potential N-linked glycosylation site at asparagine in the heavy chain variable region, wherein the potential N-linked glycosylation site in both heavy chain variable regions is removed. Method of altering the glycosylation pattern of an antibody is known in the art, such as those described in U.S. Pat. No. 6,933,368, the disclosure of which incorporated herein by reference.

### IV. Pharmaceutical Compositions, Kits, and Articles of Manufacture

In other aspects, the present application provides a composition comprising any one of the anti-CTLA4 antibodies described herein. In some embodiments, the composition is a pharmaceutical composition comprising the anti-CTLA4 antibody and a pharmaceutically acceptable carrier. The compositions can be prepared by conventional methods known in the art.

The term “pharmaceutically acceptable carrier” refers to any inactive substance that is suitable for use in a formulation for the delivery of an active agent (e.g., the anti-CTLA4 antibody). A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like) dextrose, vegetable oils (such as olive oil), saline, buffer, buffered saline, and isotonic agents such as sugars, polyalcohols, sorbitol, and sodium chloride. The compositions may be in any suitable forms, such as liquid, semi-solid, and solid dosage forms. Examples of liquid dosage forms include solution (e.g., injectable and infusible solutions), microemulsion, liposome, dispersion, or suspension. Examples of solid dosage forms include tablet, pill, capsule, microcapsule, and powder. A particular form of the composition suitable for delivering an anti-CTLA4 antibody is a sterile liquid, such as a solution, suspension, or dispersion, for injection or infusion. Sterile solutions can be prepared by incorporating the antibody in the required amount in an appropriate carrier, followed by sterilization microfiltration. Generally, dispersions are prepared by incorporating the antibody into a sterile vehicle that contains a basic dispersion medium and other carriers. In the case of sterile powders for the preparation of sterile liquid, methods of preparation include vacuum drying and freeze-drying (lyophilization) to yield a powder of the active ingredient plus any additional desired ingredient from a previously sterile-filtered solution thereof. The various dosage forms of the compositions can be prepared by conventional techniques known in the art.

The relative amount of an anti-CTLA4 antibody included in the composition will vary depending upon a number of factors, such as the specific anti-CTLA4 antibody and carriers used, dosage form, and desired release and pharmacodynamic characteristics. The amount of an anti-CTLA4 antibody in a single dosage form will generally be that amount which produces a therapeutic effect, but may also be a lesser amount. Generally, this amount will range from about 0.01 percent to about 99 percent, from about 0.1 percent to about 70 percent, or from about 1 percent to about 30 percent relative to the total weight of the dosage form.

In addition to the anti-CTLA4 antibody, one or more additional therapeutic agents may be included in the composition. Examples of additional therapeutic agents are described herein in the “Methods of Treatment” section. The suitable amount of the additional therapeutic agent to be included in the composition can be readily selected by a person skilled in the art, and will vary depending on a number of factors, such as the particular agent and carriers used, dosage form, and desired release and pharmacodynamic characteristics. The amount of the additional therapeutic agent included in a single dosage form will generally be that amount of the agent, which produces a therapeutic effect, but may be a lesser amount as well.

In some embodiments, there is provided an article of manufacture comprising materials useful for the treatment of a cancer. The article of manufacture can comprise a container and a label or package insert on or associated with the container. Suitable containers include, for example, bottles, vials, syringes, etc. The containers may be formed from a variety of materials such as glass or plastic. Generally, the container holds a composition, which is effective for treating a cancer, described herein, and may have a sterile access port (for example, the container may be an intravenous solution bag or a vial having a stopper pierceable by a hypodermic injection needle). Package insert refers to instructions customarily included in commercial packages of therapeutic products that contain information about the indications, usage, dosage, administration, contraindications and/or warnings concerning the use of such therapeutic products. In some embodiments, the package insert indicates that the composition is used for treating a cancer. The label or package insert may further comprise instructions for administering the composition to a patient.

Additionally, the article of manufacture may further comprise a second container comprising a pharmaceutically acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for treatment of a cancer described herein, optionally in combination with the articles of manufacture. Kits of the present application include one or more containers comprising any one of the compositions described herein (or unit dosage form and/or article of manufacture). In some embodiments, the kit further comprises other agents (e.g., one or more additional therapeutic agents) and/or instructions for use in accordance with any of the methods described herein. The kit may further comprise a description of selection of individuals suitable for treatment. Instructions supplied in the kits of the present application are typically written instructions on a label or package insert (e.g., a paper sheet included in the kit), but machine-readable instructions (e.g., instructions carried on a magnetic or optical storage disk) are also acceptable.

For example, in some embodiments, there is provided a kit comprising a pharmaceutical composition comprising any one of the anti-CTLA4 antibodies described herein and a pharmaceutically acceptable carrier; and instructions for administering the pharmaceutical composition to a subject having a cancer. In some embodiments, the kit further comprises a pharmaceutical composition comprising an additional therapeutic agent, such as a chemotherapeutic agent. In some embodiments, the kit comprises one or more assays or reagents thereof for determining a level of one or more biomarkers described herein (e.g., CD8+ T cells, CD4+ T cells, CD8+Tₑₘ cells, CD4+Tₑₘ cells, T_{reg} cells, a ratio of CD8+Tₑₘ cells to T_{reg} cells, a ratio of CD4+Tₑₘ cells to T_{reg} cells, NK cells, B cells).

The kits of the present application are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The containers may be unit doses, bulk packages (e.g., multi-dose packages) or sub-unit doses. Kits may also include multiple unit doses of the pharmaceutical compositions and instructions for use and packaged in quantities sufficient for storage and use in pharmacies, for example, hospital pharmacies and compounding pharmacies.

### EXAMPLES

The invention can be further understood by reference to the following examples, which are provided by way of illustration and are not meant to be limiting.

### Example 1. Phase 1b, Open-Label, Dose Escalation Study of TY21580, TY21580 Combined with Toripalimab (Anti-PD-1 Antibody) in Patients with Advanced/Metastatic Solid Tumors

The following example describes an ongoing phase 1b clinical trial to assess the safety and tolerability of TY21580 monotherapy and TY21580 combined with Toripalimab (see ClinicalTrials.gov Identifier: NCT04501276).

Objectives. Primary objectives of the study are to assess the safety and tolerability of TY21580 administered intravenously (IV) at escalating dose levels, TY21580 alone and in combination with toripalimab in adults with advanced/metastatic solid tumors, and to determine the maximum tolerated doses (MTDs) and recommended Phase 2 doses (RP2Ds) for monotherapy TY21580 and TY21580 in combination with toripalimab. Secondary objectives of the study are to assess the pharmacokinetic (PK) profile of TY21580, as monotherapy or in combination with toripalimab; to assess the dose proportionality of key PK parameters (area under the time concentration curve [AUC], maximum [peak] plasma concentration [Cmax], etc.) for TY21580; to assess the immunogenicity of TY21580, as monotherapy or in combination with toripalimab; to characterize the relationship between immunogenicity (anti-drug antibody [ADA] positivity) and PK, safety, and efficacy parameters for TY21580; to assess the preliminary antitumor activity of TY21580, as monotherapy and in combination regimens.

Methodology. This is a Phase 1b/2, open-label, dose escalation study of TY21580 monotherapy, TY21580 in combination with toripalimab in patients with advanced/metastatic solid tumors. The anticipated total number of patients will be up to approximately ˜82 patients.

The dose escalation consists of 2 parts:
Part A—Dose escalation of TY21580 monotherapy once every 3 weeks (Q3W).
Part B—Dose escalation of TY21580 Q3W combined with 240 mg toripalimab Q3W.

A treatment cycle is 21 days with one IV dose of TY21580, TY21580 combined with toripalimab, administered on Day 1 of the cycle. For patients in the TY21580-toripalimab combination regimens, toripalimab will be administered approximately 30 mins (±5 min) after completion of TY21580 IV infusion. The number of treatment cycles of TY21580 might be adjusted with emerging safety and PK/PD (eg, biomarker, efficacy) data. DLTs will be assessed by SRC after all patients enrolled in the dose level have been followed for at least 21 days after the first dose of a given regimen (DLT observation period). During the study, patients will be evaluated for safety and toxicity, PK, immunogenicity, ORR, DOR, PFS, OS and pre-specified biomarkers per protocol.

Part A (TY21580 monotherapy dose escalation) will utilize an Accelerated Titration Design (ATD) in the lower dose levels (DL1 and DL2) followed by a traditional 3+3 dose escalation design or mTPI design in the higher dose levels until the RP2D is determined. The starting dose will be DL1 (0.003 mg/kg).

The study will be conducted using 10 potential dose levels administered by IV infusion as shown in Table 1.

TABLE 1
Potential TY21580 Monotherapy Dose Escalation Levels

| | Dose | Dose, | Percent Increase from |
|---|---|---|---|
| Design | Level (DL)* | mg/kg | Previous Dose |
| Accelerated | DL −1 | 0.001 | — |
| titration design | DL1 | 0.003 | 300% |
| | DL2 | 0.01 | 333% |
| Traditional | DL3 | 0.03 | 300% |
| 3 + 3 design** | DL4 | 0.1 | 333% |
| | DL5 | 0.3 | 300% |
| | DL6 | 1.0 | 333% |
| | DL7 | 3.0 | 300% |
| | DL8 | 6.0 | 200% |
| | DL9 | 10.0 | 166% |
| | DL10 | 15.0 | 150% |

*The proposed doses, schedule(s), and PK time points may be reconsidered and amended during the study based on safety data and observed systemic exposures and as determined by the SRC. DL −1 is a lower titration dose to be used in the event of DLTs or clinically significant grade ≥2 toxicity AEs in DL1.
**If a single patient experiences a DLT or two ≥Grade 2 drug-related toxicities during the 21-day DLT evaluation period, traditional 3 + 3 dose escalation criteria will apply for that dose level with enrollment of additional patients. All subsequent dose levels will then follow traditional 3 + 3 dose escalation criteria.

During the ATD phase, 1 patient per dose level will be treated. If the patient experiences a DLT or two Grade ≥2 drug-related toxicity (as agreed upon by Investigator and Sponsor), the dose level will be expanded according to a 3+3 design.

From DL3 (0.03 mg/kg) onwards, dose escalation will follow a traditional 3+3 design with 3 or 6 patients treated at each dose level, depending upon the incidence of DLTs. Initially, 3 patients will be enrolled into the dose level with the sentinel patient treated at least 24 hours before the subsequent patients.

For the TY21580-toripalimab combination regimens: A modified Toxicity Probability Interval (mTPI) design with a target DLT rate of approximately 30% will be applied for dose escalation and confirmation to determine a RP2D for TY21580 in combination with toripalimab. The dose escalation includes the dose escalation cohorts as shown in Table 2.

TABLE 2
Dose-Escalation Cohorts Per mTPI Design

| | Dose | TY21580 | Toripalimab |
|---|---|---|---|
| Design | level (DL) | (Q3W) | (Q3W) |
| mTPI | DL1^{a} | SRC determined combo dose | 240 mg |
| design | | (≤6 mg/kg) | |
| | DL2 | SRC determined combo dose + 1 | 240 mg |
| | DL3 | ≤15 mg/k^{b} | 240 mg |

^{a}The SRC approved 6 mg/kg as the starting dose for TY21580 in combination arms.
^{b}DL3 is optional for the TY21580 + toripalimab combination.

The SRC approved 6 mg/kg as the starting dose for TY21580 in combination arms. Starting from the SRC-determined combination dose, dose escalation will continue to the next dose level (eg, 10 mg/kg) depending on the clearance of TY21580 monotherapy, until the RP2D of the combination is determined. The maximum dose of TY21580, to be administered in its combination therapy with toripalimab, will not exceed the RP2D determined for TY21580 monotherapy. A de-escalation dose (DL-1) of TY21580 is available if the starting dose (DL1) of TY21580 in the combination setting with toripalimab is deemed not tolerable. All dose escalation and de-escalation decisions will be based on the occurrence of DLTs. DLTs will be assessed by the SRC after all patients enrolled in the dose level have been followed for at least 21 days after the first dose of a given regimen (DLT observation period). The Medical Monitor will be contacted and will review any adverse events, Day 8 and 15 laboratories obtained from the first patient treated at that dose level once available.

The safety and tolerability of each dose level will be assessed by the SRC after all patients enrolled in the dose level have been followed for at least 21 days after the first dose of the TY21580-toripalimab combination regimen (DLT observation period). Once either the MTD or maximum administered dose (MAD) are reached, the RP2D will be determined. The RP2D will be defined based on the observation of either MTD in a dose level cohort, or by the MAD dose in the absence of MTD or DLT observation, to include dose levels below the MTD or MAD, or intermediate between the pre-specified dose levels based on an overall assessment of all safety data, as well as all available PK and pharmacodynamic data, and documented objective response observations. RP2D will be a pharmacologically active dose.

Patients who discontinue treatment due to intolerable AEs related to TY21580, TY21580 combination with toripalimab regimen will be followed-up until the AEs have returned to Grade 0 or 1, or become stable, or until the patient receives new non-protocol treatment. The Sponsor may also decide to terminate the study at any time.

Investigational Product, Dosage and Mode of Administration. TY21580 is administered IV over a period of 60-90 mins (±15 mins) Q3W at the doses specified in Table 1 for monotherapy. Toripalimab is administrated according to the package insert 240 mg, Q3W. For TY21580-toripalimab combination regimens, toripalimab will be administered 30 mins (±5 min) after TY21580 end of infusion.

Duration of Treatment. If the Investigator considers that continued treatments could benefit the patient, study treatment of monotherapy TY21580 (Q3W) or TY21580 combination regimens (Q3W for each drug in either TY21580-toripalimab regimen) will continue up to 2 years, or until disease progression and/or unacceptable toxicity, or withdrawal of informed consent, whichever occurs first.

Safety Evaluations. Safety assessments will be carried out during specified periodic physical examination findings, vital signs, ECOG performance status, laboratory variables (eg, liver tests/monitoring, hematology, coagulation tests, serum chemistry, urine tests and pregnancy test), ECG, and AEs. AEs are graded according to the NCI CTCAE v5.0. Investigators and site personnel will be responsible for properly documenting and reporting AEs/SAEs. Prior to dose escalation, the SRC will review the safety data from the current level after all patients have completed the first cycle of a minimum of 21 days, to determine whether escalating (or de-escalating) to next dose level should proceed. These decisions will be documented.

Efficacy Evaluations. Tumor assessments for response/progression will be performed at baseline, and every 6 weeks (±1 week) for the first 12 weeks, then assessments will be carried out every 9 weeks (±1 week) for the remaining treatment duration thereafter, until disease progression or death, treatment/study discontinuation due to treatment toxicity, loss to follow-up, withdrawal of consent, start of new cancer treatment, or study completion/closure, whichever occurs first. The exploratory evaluation of efficacy will be based on tumor assessment by the Investigator per RECIST v1.1 and/or iRECIST. All discontinued patients who received study drug will be included in efficacy analysis of the intention-to-treat population. A subgroup exploratory analysis will be conducted in those who have received at least 1 dose of TY21580 treatment and have at least 1 tumor assessment among total discontinuation for all reasons.

Pharmacokinetic and Immunogenicity Evaluations. Blood samples will be collected from all patients during the first cycle to determine the serum concentration of TY21580, and toripalimab in the corresponding drug dosed patients. PK parameters will be monitored more intensively during the first treatment cycle. Starting from the second to the fourth administration, and every 2 cycles thereafter if treatment will continue beyond 4 cycles, serum concentration of drug antibodies will be collected within 30 minutes before the administration of the drug (Ctrough) and at the end of infusion. The PK sampling time points may be adjusted based on cumulative data.

Blood samples for ADAs against each drug antibody in the corresponding drug dosed patients, will be collected at pre-dose of cycles 1, 2, 3, 4, and every 4 cycles thereafter if treatment will continue beyond 4 cycles. Additionally, ADA samples will be collected at the end of study and the last follow-up visit if feasible. Neutralization activity will be evaluated if ADA is positive.

### Monotherapy Results

Interim results of the study are described below.

### General Observations

46 patients were enrolled into the Phase Ib study and treated with TY21580 alone at different dose escalation cohorts (i.e., 1 mg/kg, 3 mg/kg, 6 mg/kg, 10 mg/kg, 15 mg/kg), including 10 with ovarian/fallopian tube cancer, 6 with pancreatic cancer, 5 with mCRC, 4 with renal cell carcinoma, 4 with endometrial cancer, 2 with melanoma and 2 with urothelial cancer, others cancer types included soft tissue sarcoma, breast cancer, esophageal cancer, HNSCC, etc. The median age was 62; 62% received ≥3 prior lines of therapies, and 31% progressed after prior IO therapies. Only 1 DLT [Grade (G) 4 hyperglycemia] was observed (10 mpk), and MTD was not reached. Only 3 patients show G3 or above treatment-related AEs (TRAEs), one patient was reported both grade 3 and grade 4; common TRAEs (>10%) consisted of diarrhea, pruritis, and fatigue. 34 patients had at least one tumour measurement after baseline, 10 had stable disease, 1 patient with Kaposi sarcoma (HIV unrelated) had PR.

TY21580 demonstrated favourable safety and tolerability profiles at doses up to 15 mg/mg (Table 3 and Table 4). No MTD identified for monotherapy up to 15 mg/kg Q3W, and with only one DLT at 10 mg/kg

Upon treatment with TY21580, significant increases in absolute T cell and N cell levels, and decreases in the percentage of T_{reg} cells correlates with favorable clinical outcomes. It is anticipated that TY21580 has the potential to overcome the limitations of existing anti-CTLA-4 checkpoint inhibitors on the market, and extend the market potential beyond current anti-CTLA-4 inhibitors in both monotherapy and combination settings. The anti-CTLA-4 therapeutics described herein may improve the clinical benefits by expanding clonal diversity, infiltrating into cold tumors, and treating patients resistant/refractory to current immuno-therapies. Specific examples of treating individual subjects in the study are described below.

TABLE 3
Treatment-Related Adverse Event (TRAE) of TY21580 monotherapy

| | Dose level | | | | | |
|---|---|---|---|---|---|---|
| | 1 mg/kg | 1 mg/kg | 3 mg/kg | 6 mg/kg | 10 mg/kg | 15 mg/kg |
| Summary | (N = 11) | (N = 3) | (N = 4) | (N = 6) | (N = 19) | (N = 3) |
| Subjects with | 3 (27.3) | 1 (33.3) | 3 (75.0) | 5 (83.3) | 12 (63.2) | 3 (100.0) |
| any TRAE | | | | | | |
| Grade 1 | 3 (27.3) | 1 (33.3) | 1 (25.0) | 3 (50.0) | 6 (31.6) | 0 |
| Grade 2 | 0 | 0 | 2 (50.0) | 2 (33.3) | 3 (15.8) | 3 (100.0) |
| Grade 3 | 0 | 0 | 0 | 0 | 2 (10.5) | 0 |
| Grade 4 | 0 | 0 | 0 | 0 | 1 (5.3) | 0 |
| Grade 5 | 0 | 0 | 0 | 0 | 0 | 0 |

TABLE 4
G3/G4 Serious Adverse Reaction (SAR) of TY21580 monotherapy

| | | Number of subjects exposed | | |
|---|---|---|---|---|
| | | to TY21580 monotherapy(n = 46) | | |
| | | | Occurrence of | Occurrence of life |
| | | All SARs | fatal SARs | threatening SARs |
| System organ class | SARs | N(%) | N(%) | N(%) |
| Gastrointestinal | Immune-mediated | 1(2.2) | 0(0) | 0(0) |
| disorders | enterocolitis (G3) | | | |
| Investigations | Aspartate aminotransferase | 1(2.2) | 0(0) | 0(0) |
| | increased(G3) | | | |
| Metabolism and | Hyperglycaemia(G4) | 1(2.2) | 0 | 0 |
| nutrition disorders | | | | |
| Skin and | Rash maculo-papular(G3) | 1(2.2) | 0 | 0 |
| subcutaneous tissue | | | | |
| disorders | | | | |

### Subject 1

A 74-year-old male with renal cell carcinoma who relapsed on nivolumab and is enrolled in the 10 mg/kg cohort. CD8+ T cells increased in this subject after the 1st cycle of treatment showing that TY21580 is highly active for triggering T cell activation.

### Subject 2

A 63-year-old white female with clear cell carcinoma and underwent bilateral salpingo-oophorectomy, this patient became resistant to platinum-based chemotherapy. After being treated with four cycle of TY21580 monotherapy (6 mg/kg), the sum of the size of the targeted lesions decreased in the following two tumour assessments (Table 5).

### Subject 3

A 77-year-old male with pancreatic cancer in the 10 mg/kg cohort who has undergone three previous therapies. This subject has two target lesions, one in the pancreas and one in the liver with baseline measurements of 35 mm for the pancreas lesion and 15 mm for the liver lesion. The first tumor assessment was performed on this patient after two cycles of TY21580 treatment. This assessment shows the pancreas lesion has shrunk to 29 mm and the liver lesion has shrunk to 10 mm, reflecting a 22% reduction in target lesion size (Table 5).

### Subject 4

A 69-year-old male with recurrent Kaposi sarcoma unrelated with HIV, had previously underwent multiple surgeries for both left and right feet and had been treated with etoposide as systemic chemotherapy. After being treated with two cycles of TY21580 monotherapy (15 mg/kg), the target lesion on the left foot completely disappeared, and the tumor assessment is PR (partial response, Table 5).

TABLE 5
Clinical response and anti-tumour efficacy of TY21580 monotherapy

| | | | Target Lesion (Tumor Size/mm) | | | Non-Target Lesion | | | |
|---|---|---|---|---|---|---|---|---|---|
| Subject | Dose | Tumor | | Base- | Tumor | Base- | Tumor | New | Over- |
| No. | cohort | Type | | line | Shrinkage | line | Shrinkage | Lesion | all |
| Subject | 6 | Endo- | Sum | 40 mm | 34 mm (−15%) | Present | Present | No | SD |
| 2 | mg/kg | metrial | TL1- Lung | 19 mm | 14 mm (−26%) | | | | |
| | | carcinoma | TL2- Lung | 21 mm | 20 mm (−5%) | | | | |
| Subject | 10 | Pancreatic | Sum | 50 mm | 39 mm (−22%) | Present | Dis- | No | SD |
| 3 | mg/kg | cancer | TL 1- Pancreas | 35 mm | 29 mm (−17%) | | appeared | | |
| | | | TL2 -Liver | 15 mm | 10 mm (−33%) | | | | |
| Subject | 15 | Kaposi | Sum | 10 mm | Disappeared | Present | Present | No | PR |
| 4 | mg/kg | sarcoma | TL1-Left foot | 10 mm | Disappeared | | | | |

### Combination Therapy Results

Interim results of the study are described below.

### General Observations

So far nine patients have received TY21580 and Toripalimab combination therapy (Table 6 and Table 7). The median age was 59; 11% received >3 prior lines of therapies, and none had prior IO therapies. At 6 mpk TY21580 and Toripalimab combination, two pts developed DLTs (2/3; G3 myocarditis & G3 diarrhea). At 3 mpk of TY21580 and Toripalimab combination, 1 DLT (1/6, G3 diarrhea) was observed, and five pts are still on treatment (2-5 cycles).

3 patients' tumour measurement were available and 1 patient with recurrent platinum-refractory HNSCC has a confirmed complete response (CR), 2 patients had stable disease and had reduction of the sum of the size of target lesions.

Based on initial safety and efficacy for combination study, TY21580 (3 mg/kg, Q3W) combined with Toripalimab (240 mg, Q3W) continuous dosing showed a manageable safety profile and encouraging efficacy including CR, supporting its further clinical development,

TABLE 6
Treatment-Related Adverse Event (TRAE) of
TY21580 and Toripalimab Combination therapy

TY21580 + Toripalimab TRAE (N, %)

| | TY21580 3 mg/kg | TY21580 6 mg/kg |
|---|---|---|
| N | 6 | 3 |
| G1 | 4 (66.7%) | 0 (0%) |
| G2 | 0 (0%) | 0 (0%) |
| G3 | 2 (33.3%) | 3 (100%) |
| G4 | 0 (0%) | 0 (0%) |

TABLE 7
G3/G4 Serious Adverse Reaction (SAR) of TY21580
and Toripalimab Combination therapy

| | | Number of subjects exposed to TY21580 | | |
|---|---|---|---|---|
| | | combined with Toripalimab (N) = 9 | | |
| | | | Occurrence of | Occurrence of life- |
| | | All SARs | fatal SARs | threatening SARs |
| System organ class | SARs | N (%) | N (%) | N (%) |
| Cardiac disorders | Immune-mediated | 1(11) | 0 | 0 |
| | myocarditis(G3) | | | |
| Gastrointestinal disorders | Diarrhoea(G3) | 2(22) | 0 | 0 |
| Investigations | Lipase | 1(11) | 0 | 0 |
| | increased(G3) | | | |

Subject 1—combination Arm

A 65-year-old male with recurrent head and neck squamous carcinoma after comprehensive multi-disciplinary treatment comprising surgery, radiotherapy and chemotherapy, had complete response (CR) after 2 cycles of treatment with TY21580 (3 mg/kg, Q3W) combined with Toripalimab (240 mg, Q3W), and the CR was confirmed in the following tumor assessments (Table 8).

### Subject 2—Combination Arm

A 69-year-old female was diagnosed with angiosarcoma, most recent metastatic disease documented with right axillary nodes, multiple metastatic lesions in liver (approximately 30) and spleen (approximately 20), widespread bony metastasis, she was previously heavily treated, Prior therapies included doxorubicin, paclitaxel and Eribulin.

The patient commenced on TY21580 (3 mg/kg, Q3W) combined with Toripalimb (240 mg, Q3W), the sum of the size of the target lesions reduced remarkably, with 25% decrease on the second tumor assessment (Table 8).

TABLE 8
Clinical response and anti-tumor efficacy of TY21580 and Toripalimab combination therapy

| | | Target Lesion (Tumor Size/ mm) | | | Non-Target Lesion | | | |
|---|---|---|---|---|---|---|---|---|
| Subject | Tumor | | Base- | After | Base- | Tumor | New | Over- |
| No. | Type | | line | Treatment | line | Shrinkage | Lesion | all |
| Subject | HNSCC | Sum | 65 mm | 8 mm | Present | Disappeared | No | CR |
| 1 | | TL1 | 32 mm | Disappeared | | | | |
| | | Right mandibular | | | | | | |
| | | TL2 | 18 mm | Disappeared | | | | |
| | | Right | | | | | | |
| | | submandibular | | | | | | |
| | | TL3 | 15 mm | 8 mm (−46%) | | | | |
| | | Left submandibular | | | | | | |
| Subject | Angio- | Sum | 125 mm | 94 mm (−25%) | Present | Present | No | SD |
| 2 | sarcoma | TL1-Liver | 32 mm | 18 mm (−44%) | | | | |
| | | TL2-Liver | 27 mm | 16 mm (−41%) | | | | |
| | | TL3-Spleen | 32 mm | 39 mm (22%) | | | | |
| | | TL4-Spleen | 34 mm | 21 mm (−38%) | | | | |

### Example 2: TY21580 PK Comparison with Ipilimumab (Ipi) Supports 3 mg/Kg Q3W and Q6W Dosing in Combination Treatment Settings (with Anti-PD-1 mAbs)

### Methods

TY21580 serum interim PK was obtained and using validated PK assays. Noncompartmental analysis (NCA, Phoenix® WinNonlin®) and population PK (popPK) analysis by Phoenix NLME™ (Phoenix WinNonlin V8.3, Certara) were used for analyzing TY21580 interim PK data.

### PK and Dosing Assessment

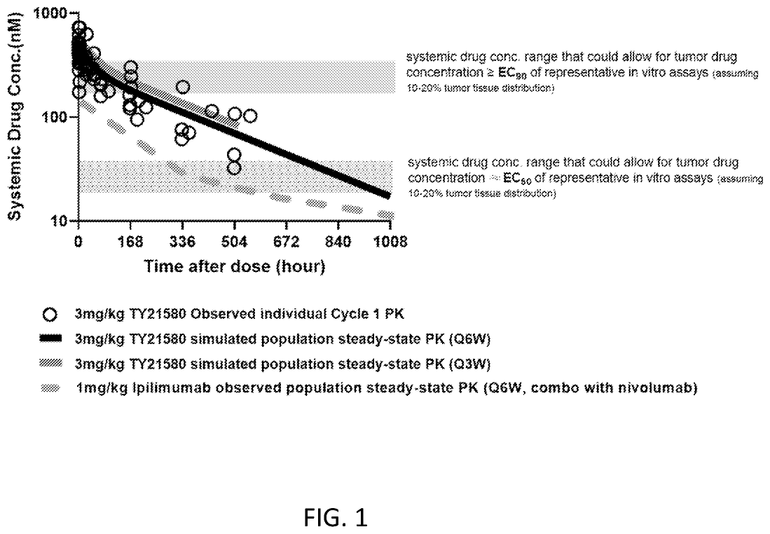

Population PK modeling was conducted for TY21580 and FIG. 1 shows the observed Cycle 1 individual PK of TY21580 after 3 mg/kg dosing and model-predicted TY21580 population PK at steady-state (Q3W or Q6W repeat dosing at 3 mg/kg) against Ipilimumab's observed PK at steady state (repeat dosing at 1 mg/kg). Comparing with Ipilimumab's published systemic PK and head-to-head comparison of in vitro binding and functional data, TY21580 is expected to achieve greater receptor occupancy (RO) and target engagement (TE) at 3 mg/kg Q3W and Q6W dosing compared with Ipilimumab's 1 mg/kg Q6W dosing currently approved for combination treatment with Nivolumab for a number of cancer indications (reference: https://packageinserts.bms.com/pi/pi_yervoy.pdf).

With Q6W dosing of 3 mg/kg of TY21580, the calculated tumor tissue RO (CTLA-4, factoring antibody tumor partition of 10-20% compared with systemic PK) based on in vivo TY21580 PK and in vitro binding and CD80/CD86 ligand blocking data, is expected to be >90% for ˜7 days (RO is >EC₉₅ at Cₘₐₓ), and >50% for ˜6 weeks post dosing. When TY21580 is dosed Q3W, due to greater PK accumulation compared with Q6W dosing, the RO coverage at steady state is greater. In contrast, the calculated tumor tissue RO based on mean in vivo Ipilimumab PK (1 mg/kg Q6W dosing, steady-state) and Ipilimumab's in vitro binding/ligand blocking data is expected to be <90% at Cₘₐₓ, and drop below 50% ˜3 weeks post dosing (e.g., using the same antibody tumor partition of 10-20%). Further noncompartmental analysis (NCA) suggested that the AUC_{tau,SS} (area under the concentration-time curve during each dosing interval at steady state) is ˜2-fold or greater for 3 mg/kg of TY21580 (Q6W or Q3W repeat dosing), when compared with Ipilimumab (1 mg/kg Q6W repeat dosing). Similarly, C_{max,SS} (maximum drug concentration at steady state) is ˜3-fold for 3 mg/kg of TY21580 (Q6W or Q3W repeat dosing), when compared with Ipilimumab (1 mg/kg Q6W repeat dosing).

Based on the integrated understanding of the mechanism of actions (MOAs) of TY21580 and Ipilimumab that drive clinical efficacy, the superior clinical safety profiles of TY21580 as monotherapy and in combination treatment settings (e.g., with anti-PD-1 mAbs) allow TY21580 to be dosed significantly higher (e.g., 3× in terms of mg/kg) and more frequently/continuously than Ipilimumab when combined with anti-PD-1 in a number of approved cancer indications (e.g., Ipilimumab dosed at 1 mg/kg Q6W repeat dosing or 3 mg/kg Q3W dosing for the first 4 cycles), reaching RO values at the site of action (e.g., ≥90%) that can drive greater pharmacodynamic (PD) effects.

Taken together, this analysis supports that TY21580 3 mg/kg Q3W and/or Q6W continuous dosing when combined with anti-PD-1 (or PD-L1) mAbs may result in greater therapeutic benefit compared with approved similar regimens (e.g., Ipilimumab+nivolumab) based on integrated PK/PD assessment.

### Example 3. Treatment of Metastatic Endometrial Cancer with TY21580

A 44-year-old female was diagnosed with endometrial carcinoma, microsatellite instability high (MSI-H). The most recent metastatic disease documented was with right upper trachea lymph node metastatic lesion (approximately 19 mm) in lung and left clavicle lymph node metastatic lesion (approximately 31 mm) in lung. She was previously heavily treated, prior therapies included docetaxel, cisplatin, and EZH1/EZH2 inhibitor. The patient commenced on TY21580 (10 mg/kg, Q3W) monotherapy. Following treatment, the sum of the size of the target lesions reduced remarkably on the first tumor assessment. As shown in Table 9, the best response occurred on the third tumor assessment, with 50% decrease based on sum of target lesions of screening.

TABLE 9
Anti-tumor efficacy of TY21580 in treating patient with metastatic endometrial cancer

| | | Baseline | 6 weeks | 12 weeks | 21 weeks | 30 weeks |
|---|---|---|---|---|---|---|
| Target | TL1 - | 19 mm | 17 mm | 10 mm | 10 mm | 9 |
| Lesion | Lymph | | | | | |
| | Node | | | | | |
| | TL2- | 31 mm | 27 mm | 19 mm | 15 mm | 16 |
| | Lymph | | | | | |
| | Node | | | | | |
| | Sum | 50 mm | 44 mm | 29mm | 25mm | 25mm |
| | | | (−12%) | (−42%) | (−50%) | (−50%) |
| Non- | Multiple | Present | Present | Present | Present | Present |
| Target | | | | | | |
| Lesions | | | | | | |
| New | | N/A | No | No | No | No |
| Lesion | | | | | | |
| Overall | | N/A | SD | PR | PR | PR |

### Example 4. Treatment of Metastatic Clear Cell Renal Cell Carcinoma with TY21580

A 56-year-old male was diagnosed with clear cell renal cell carcinoma, The most recent metastatic disease documented with right middle lower metastatic lesion (approximately 15 mm) in lung, right middle lobe metastatic lesion (approximately 16 mm) in lung, right interlobar lymph node metastatic lesion (approximately 22 mm), metastatic lesion (approximately 47 mm) in kidney, and retrocaval lymph node me metastatic lesion (approximately 15 mm). He was previously treated with Sunitinib, followed by oral PD-L1 inhibitor in development. The patient commenced on TY21580 (10 mg/kg, Q3W) monotherapy. As shown in Table 10, following treatment with TY21580, the sum of the size of the target lesions reduced remarkably, with 41% decrease on the third tumor assessment.

TABLE 10
Anti-tumor efficacy of TY21580 in treating patient with
metastatic clear cell renal cell carcinoma.

| | | Baseline | 6 weeks | 12 weeks | 21 weeks | 30 weeks | 39 weeks | 48 weeks |
|---|---|---|---|---|---|---|---|---|
| Target | TL1- | 15 mm | 11 mm | 11 mm | 9 mm | 9 mm | 9 mm | 9 mm |
| Lesion | Lung RLL | | | | | | | |
| | TL2 - | 16 mm | 14 mm | 14 mm | 13 mm | 13 mm | 13 mm | 13 mm |
| | Lung | | | | | | | |
| | RML | | | | | | | |
| | TL3 - LN | 22 mm | 14 mm | 14 mm | 14 mm | 14 mm | 14 mm | 14 mm |
| | TL-4- R | 47 mm | 47 mm | 30 mm | 27 mm | 27 mm | 27 mm | 27 mm |
| | kidney | | | | | | | |
| | TL-5- LN | 15 mm | 10 mm | 7 mm | 5 mm | 5 mm | 5 mm | 5 mm |
| | Sum | 115 mm | 96 mm | 76 mm | 68 mm | 68 mm | 68 mm | 68 mm |
| | | | (−17%) | (−34%) | (−41%) | (−41%) | (−41%) | (−41%) |
| Non- | Multiple | Present | Present | Present | Present | Present | Present | Present |
| Target | | | | | | | | |
| Lesions | | | | | | | | |
| New | | N/A | No | No | No | No | No | No |
| Lesion | | | | | | | | |
| Overall | | N/A | SD | PR | PR | PR | PR | PR |

### Example 5. Treatment of Metastatic HSNCC with TY21580 in Combination with Toripalimab

A 64-year-old male was diagnosed with HPV negative recurrent head and neck squamous cell carcinoma (HNSCC). The most recent metastatic disease documented with right mandibular metastatic lesions in liver (approximately 30) and right submandibular (approximately 20), lymph nodes metastasis. His prior therapies included right modified cervical lymph node dissection followed by adjuvant radiotherapy (local therapy); and concurrent chemoradiotherapy, including weekly cisplatin. The patient commenced on TY21580 (3 mg/kg) combination with Toripalimab 240 mg, Q3W. As shown in Table 11, the sum of the size of the target lesions reduced remarkably, with durable complete response observed since the first tumor assessment,

TABLE 11
Anti-tumor efficacy of TY21580 in combination with Toripalimab in
treating patient with metastatic HSNCC

| | | Base- | 6 | 12 | 21 | 30 | 39 | 48 | 57 |
|---|---|---|---|---|---|---|---|---|---|
| | | line | weeks | weeks | weeks | weeks | weeks | weeks | weeks |
| Target | TL1 - | 32 mm | Disap- | Disap- | Disap- | Disap- | Disap- | Disap- | Disap- |
| lesion | right | | peared | peared | peared | peared | peared | peared | peared |
| | mandibular | | | | | | | | |
| | TL2 - | 18 mm | Disap- | Disap- | Disap- | Disap- | Disap- | Disap- | Disap- |
| | right | | peared | peared | peared | peared | peared | peared | peared |
| | submandibular | | | | | | | | |
| | TL3- LN | 15 mm | 8 mm | 8 mm | 5 mm | 5 mm | 5 mm | 5 mm | 5 mm |
| | Sum | 65 mm | 8 mm | 8 mm | 5 mm | 5 mm | 5 mm | 5 mm | 5 mm |
| Non- | 3 | Present | Disap- | Disap- | Disap- | Disap- | Disap- | Disap- | Disap- |
| target | | | peared | peared | peared | peared | peared | peared | peared |
| lesion | | | | | | | | | |
| New | | | No | No | No | No | No | No | No |
| Lesion | | | | | | | | | |
| Overall | | | CR | CR | CR | CR | CR | CR | CR |

### Example 6. Treatment of Metastatic Colorectal Cancer with TY21580 in Combination with Toripalimab

A 50-year-old female was diagnosed with microsatellite stable (MSS) colorectal cancer.

The most recent metastatic disease documented with extensive lymph node metastasis in right paraaortic and common iliac nodes (approximately 45). He was previously heavily treated with anti-cancer therapies including MFOLFOXIRI, bevacizumab, encorafenib, cetuximab, and TAS102. The patient commenced on TY21580 3 mg/kg, Q6W; combination with Toripalimab 240 mg, Q3W. As shown in Table 12, the sum of the size of the target lesions reduced remarkably, with 32% decrease of partial response with initial tumor assessment.

TABLE 12
Anti-tumor efficacy of TY21580 in combination with Toripalimab
in treating patient with metastatic colorectal cancer

| | | Baseline | 6 weeks |
|---|---|---|---|
| Target lesions | TL1-LN right | 25 mm | 15 mm |
| | paraaortic) | | |
| | TL2 -LN common | 20 mm | 15 mm |
| | iliac) | | |
| | Sum (percent | 45 mm | 30 mm(−33%) |
| | change) | | |
| Non-target lesion | NTL1- LN | Present | Present |
| | (right lower | | |
| | paratracheal) | | |
| | NTL2- LN (right | Present | Present |
| | upper | | |
| | paratracheal) | | |
| New lesion | | N/A | No |

### Example 7. Evaluation of Immunogenicity of TY21580

TY21580 Anti-drug Antibody (ADA) method in clinical study was validated with three tiers, screening, confirmatory and titration in a GLP lab. The ADA method was used to assess the immunogenicity of TY21580 in 72 patients from clinical studies up to and including doses of 15 mg/kg TY21580. Currently, among these 72 patients from clinical study up to 15 mg/kg, only one patient showed treatment emergent ADA positive (Table 13). The treatment emergent positive rate is 1.4%. Additionally, there was no apparent impact of ADA on pharmacokinetics (PK) based on graphical assessment (e.g., time vs. PK) as well as quantitative assessment evaluating population mean PK when ADA is negative vs. individual PK at specific time points when ADA is positive.

TABLE 13
Results from ADA Assay

| TY21580 | Total | Positive TY21580 ADAs with Treatment | | | | | | | | | |
|---|---|---|---|---|---|---|---|---|---|---|---|
| Dosage (mg/kg) | | 0.003 | 0.01 | 0.03 | 0.1 | 0.3 | 11 | 3 | 6 | 10 | 15 |
| Evaluable patients | 72 | 1 | 1 | 3 | 3 | 3 | 6 | 23 | 7 | 22 | 13 |
| Negative | 69 | 1 | 1 | 1 | 3 | | 6 | 23 | 7 | 21 | 3 |
| | (95.8%) | | | | | | | | | | |
| Non-treatment-emergent | 2 | | | 2 | | | | | | | |
| positive | (2.8%) | | | | | | | | | | |
| Treatment-emergent | 1 | | | | | | | | | 1 | |
| positive | (1.4%) | | | | | | | | | | |

What is claimed is:

1. A method of treating a cancer in a subject, comprising administering to the subject an effective amount of an anti-CTLA4 antibody and an effective amount of an anti-PD-1 antibody, wherein the antibody specifically binds to an epitope comprising amino acid residues Y105 and L106 of human CTLA4 but does not comprise residue 1108, wherein the numbering of the amino acid residues is according to SEQ ID NO: 108, and wherein the anti-CTLA4 antibody is administered at a dose of from about 1 mg/kg to about 10 mg/kg.

2. The method of claim 1, wherein anti-CTLA4 antibody is administered at a dose of from about 2 mg/kg to about 5 mg/kg.

3. The method of claim 1, wherein anti-CTLA4 antibody is administered at a dose of from about 3 mg/kg to about 5 mg/kg.

4. The method of claim 1, wherein anti-CTLA4 antibody is administered at a dose of about 3 mg/kg.

5. The method of any one of claims 1-4, wherein the antibody is administered to the subject once every 3 weeks.

6. The method of any one of claims 1-4, wherein the antibody is administered to the subject once every 6 weeks.

7. The method of any one of claims 1-6, herein the cancer is resistant or refractory to a prior therapy, wherein the prior therapy is an inhibitor of CTLA4, PD-1, or a PD-1 ligand.

8. The method of claim 7, wherein the prior therapy is ipilimumab.

9. The method of any one of claims 1-8, wherein the cancer is Kaposi's sarcoma.

10. The method of any one of claims 1-8, wherein the cancer is head and neck squamous cell carcinoma (HNSCC) or angiosarcoma.

11. The method of any one of claims 1-8, wherein the cancer is pancreatic cancer.

12. The method of any one of claims 1-8, wherein the cancer is ovarian cancer.

13. The method of any one of claims 1-12, wherein the anti-PD-1 antibody is toripalimab.

14. The method of any one of claims 1-13, wherein the anti-CTLA4 antibody comprising a heavy chain variable region and a light chain variable region, wherein the heavy chain variable region of the antibody comprises an HVR-H1, an HVR-H2, and an HVR-H3, and the light chain variable region of the antibody comprises an HVR-L1, an HVR-L2, and an HVR-L3, wherein the HVR-H1 comprises an amino acid sequence according to a formula YSISSGYHWSWI (SEQ ID NO: 23), the HVR-H2 comprises an amino acid sequence according to a formula LARIDWDDDKYYSTSLKSRL (SEQ ID NO: 35), the HVR-H3 comprises an amino acid sequence according to a formula ARSYVYFDY (SEQ ID NO: 45), the HVR-L1 comprises an amino acid sequence according to a formula RASQSVRGRFLA (SEQ ID NO: 58), the HVR-L2 comprises an amino acid sequence according to a formula DASNRATGI (SEQ ID NO: 66), and the HVR-L3 comprises an amino acid sequence according to a formula YCQQSSSWPPT (SEQ ID NO: 75).

15. The method of any one of claims 1-14, wherein the anti-CTLA4 antibody comprises a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100.

16. The method of claim 15, wherein the heavy chain variable region comprises the amino acid sequence of SEQ ID NO: 87 and the light chain variable region comprises the amino acid sequence of SEQ ID NO: 100.

17. The method of claim 16, wherein the anti-CTLA4 antibody comprises a heavy chain region comprising the amino acid sequence of SEQ ID NO:126 and the light chain region comprises the amino acid sequence of SEQ ID NO:127.

18. The method of claim 16, wherein the anti-CTLA4 antibody comprises a heavy chain region comprising the amino acid sequence of SEQ ID NO:125 and the light chain region comprises the amino acid sequence of SEQ ID NO:127.

19. The method of any one of claims 1-18, wherein the subject is human.

20. A method of treating Kaposi's sarcoma in a subject, comprising administering to the subject an effective amount of an anti-CTLA4 antibody, wherein the heavy chain variable region of the antibody comprises an HVR-H1, an HVR-H2, and an HVR-H3, and the light chain variable of the antibody region comprises an HVR-L1, an HVR-L2, and an HVR-L3, wherein the HVR-H1 comprises an amino acid sequence according to a formula YSISSGYHWSWI (SEQ ID NO: 23), the HVR-H2 comprises an amino acid sequence according to a formula LARIDWDDDKYYSTSLKSRL (SEQ ID NO: 35), the HVR-H3 comprises an amino acid sequence according to a formula ARSYVYFDY (SEQ ID NO: 45), the HVR-L1 comprises an amino acid sequence according to a formula RASQSVRGRFLA (SEQ ID NO: 58), the HVR-L2 comprises an amino acid sequence according to a formula DASNRATGI (SEQ ID NO: 66), and the HVR-L3 comprises an amino acid sequence according to a formula YCQQSSSWPPT (SEQ ID NO: 75).

21. The method of claim 20, wherein the anti-CTLA4 antibody comprises a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100.

22. The method of claim 21, wherein the anti-CTLA4 antibody comprises a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100.

23. A method of treating HNSCC or angiosarcoma in a subject, comprising administering to the subject an effective amount of an anti-CTLA4 antibody, wherein the heavy chain variable region of the antibody comprises an HVR-H1, an HVR-H2, and an HVR-H3, and the light chain variable region of the antibody comprises an HVR-L1, an HVR-L2, and an HVR-L3, wherein the HVR-H1 comprises an amino acid sequence according to a formula YSISSGYHWSWI (SEQ ID NO: 23), the HVR-H2 comprises an amino acid sequence according to a formula LARIDWDDDKYYSTSLKSRL (SEQ ID NO: 35), the HVR-H3 comprises an amino acid sequence according to a formula ARSYVYFDY (SEQ ID NO: 45), the HVR-L1 comprises an amino acid sequence according to a formula RASQSVRGRFLA (SEQ ID NO: 58), the HVR-L2 comprises an amino acid sequence according to a formula DASNRATGI (SEQ ID NO: 66), and the HVR-L3 comprises an amino acid sequence according to a formula YCQQSSSWPPT (SEQ ID NO: 75).

24. The method of claim 23, wherein the anti-CTLA4 antibody comprises a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100.

25. The method of claim 23, wherein the anti-CTLA4 antibody comprises a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100.

26. The method of any one of claims 20-25, wherein the anti-CTLA4 antibody is administered as a monotherapy.

27. The method of claim 26, wherein the dose of the anti-CTLA4 antibody is from about 3 mg/kg to about 15 mg/kg once every three weeks or once every six weeks.

28. The method of claim 26, wherein the dose of the anti-CTLA4 antibody is from about 3 mg/kg to about 10 mg/kg once every three weeks or once every six weeks.

29. The method of claim 26, wherein the dose of the anti-CTLA4 antibody is from about 10 mg/kg to about 15 mg/kg once every three weeks or once every six weeks.

30. The method of any one of claims 20-25, wherein the anti-CTLA4 antibody is administered in combination with an anti-PD-1 antibody.

31. The method of claim 30, wherein the dose of the anti-CTLA4 antibody is from about 3 mg/kg to about 10 mg/kg once every three weeks.

32. The method of claim 30, wherein the dose of the anti-CTLA4 antibody is from about 3 mg/kg to about 10 mg/kg once every six weeks.

33. The method of claim 30, wherein the dose of the anti-CTLA4 antibody is from about 3 mg/kg to about 6 mg/kg once every three weeks.

34. The method of claim 30, wherein the dose of the anti-CTLA4 antibody is from about 3 mg/kg to about 6 mg/kg once every six weeks.

35. The method of claim 30, wherein the dose of the anti-CTLA4 antibody is administered at a dose of about 3 mg/kg once every three weeks.

36. The method of claim 30, wherein the dose of the anti-CTLA4 antibody is administered at a dose of about 6 mg/kg once every three weeks.

37. The method of claim 30, wherein the dose of the anti-CTLA4 antibody is administered at a dose of about 3 mg/kg once every six weeks.

38. The method of claim 30, wherein the dose of the anti-CTLA4 antibody is administered at a dose of about 6 mg/kg once every six weeks.

39. A method of treating cancer in a human patient, comprising administering to the patient an effective amount of an anti-CTLA4 antibody, wherein the heavy chain variable region of the antibody comprises an HVR-H1, an HVR-H2, and an HVR-H3, and the light chain variable region of the antibody comprises an HVR-L1, an HVR-L2, and an HVR-L3, wherein the HVR-H1 comprises an amino acid sequence according to a formula YSISSGYHWSWI (SEQ ID NO: 23), the HVR-H2 comprises an amino acid sequence according to a formula LARIDWDDDKYYSTSLKSRL (SEQ ID NO: 35), the HVR-H3 comprises an amino acid sequence according to a formula ARSYVYFDY (SEQ ID NO: 45), the HVR-L1 comprises an amino acid sequence according to a formula RASQSVRGRFLA (SEQ ID NO: 58), the HVR-L2 comprises an amino acid sequence according to a formula DASNRATGI (SEQ ID NO: 66), and the HVR-L3 comprises an amino acid sequence according to a formula YCQQSSSWPPT (SEQ ID NO: 75), and wherein the effective amount of the anti-CTLA4 antibody achieves greater than 50% receptor occupancy three weeks or six weeks following administration.

40. The method of claim 39, wherein the effective amount achieves greater than 60% receptor occupancy three weeks following administration.

41. The method of claim 39, wherein the effective amount achieves greater than 70% receptor occupancy three weeks following administration.

42. The method of claim 39, wherein the effective amount achieves greater than 80% receptor occupancy three weeks following administration.

43. The method of claim 39, wherein the effective amount achieves from about 50% to about 80% receptor occupancy three weeks following administration.

44. The method of claim 39, wherein the effective amount achieves greater than 50% receptor occupancy six weeks following administration.

45. The method of claim 39, wherein the effective amount achieves greater than 60% receptor occupancy three weeks following administration.

46. The method of claim 39, wherein the effective amount achieves greater than 70% receptor occupancy three weeks following administration.

47. The method of claim 39, wherein the effective amount achieves from about 50% receptor occupancy to about 70% receptor occupancy six weeks following administration.

48. The method of any one of claims 39-47, wherein the anti-CTLA4 antibody comprises a heavy chain variable region comprising the amino acid sequence of SEQ ID NO: 87 or a variant thereof having at least about 90% (e.g., at least about 92%, 95%, 98%, 99% or more) sequence identity to the amino acid sequence of SEQ ID NO: 87, and a light chain variable region comprising the amino acid sequence of SEQ ID NO: 100 or a variant thereof having at least about 90% (e.g., at least about 92%, 95%, 98%, 99% or more) sequence identity to the amino acid sequence of SEQ ID NO: 100.

49. The method of any one of claims 39-48, wherein the anti-CTLA4 antibody is administered in combination with one or more additional therapeutic agents.

50. The method of claim 49, wherein the one or more additional agents is an anti-PD-1 antibody.

51. The method of any one of claims 39-50, wherein the cancer is HNSCC.

52. The method of any one of claims 39-50, wherein the cancer is angiosarcoma.

53. The method of any one of claims 39-50, wherein the cancer is Kaposi's sarcoma.


SEQUENCE LISTING (139 sequences)

SEQ ID NO: 1 - protein, 11 aa, synthetic construct
  REGION 1..11: note = Synthetic Construct
  VARIANT 1: note = Xaa = Phe or Tyr
  VARIANT 5: note = Xaa = Asp or Gly
  VARIANT 7: note = Xaa = Ala, Gly or Trp
XTFSXYXIHWV

SEQ ID NO: 2 - protein, 12 aa, synthetic construct
  REGION 1..12: note = Synthetic Construct
  VARIANT 4: note = Xaa = Ser or Thr
  VARIANT 7..8: note = Xaa = His or Tyr
  VARIANT 10: note = Xaa = Ala, Asp, or Ser
YSIXSGXXWXWI

SEQ ID NO: 3 - protein, 13 aa, synthetic construct
  REGION 1..13: note = Synthetic Construct
  VARIANT 11: note = Xaa = Gly or Ser
FSLSTGGVAVXWI

SEQ ID NO: 4 - protein, 20 aa, synthetic construct
  REGION 1..20: note = Synthetic Construct
  VARIANT 3: note = Xaa = Asp or Glu
  VARIANT 5: note = Xaa = Ser or Tyr
  VARIANT 14: note = Xaa = Pro or Gln
IGXIXHSGSTYYSXSLKSRV

SEQ ID NO: 5 - protein, 21 aa, synthetic construct
  REGION 1..21: note = Synthetic Construct
  VARIANT 3: note = Xaa = Ile or Trp
  VARIANT 8: note = Xaa = Gly or Ser
  VARIANT 10: note = Xaa = Gly or Ser
  VARIANT 12: note = Xaa = Lys or Asn
IGXISPSXGXTXYAQKFQGRV

SEQ ID NO: 6 - protein, 21 aa, synthetic construct
  REGION 1..21: note = Synthetic Construct
  VARIANT 3: note = Xaa = Ala, Gly, or Ser
  VARIANT 7: note = Xaa = Ser or Tyr
  VARIANT 9: note = Xaa = Gly or Ser
  VARIANT 10: note = Xaa = Ser or Thr
VSXISGXGXXTYYADSVKGRF

SEQ ID NO: 7 - protein, 9 aa, synthetic construct
  REGION 1..9: note = Synthetic Construct
  VARIANT 3: note = Xaa = Gly, Arg, or Ser
  VARIANT 4: note = Xaa = Ala, Ile, or Tyr
  VARIANT 5: note = Xaa = Asp, Val, or Tyr
  VARIANT 6: note = Xaa = Ala, Glu, or Tyr
  VARIANT 9: note = Xaa = Ile or Tyr
ARXXXXFDX

SEQ ID NO: 8 - protein, 10 aa, synthetic construct
  REGION 1..10: note = Synthetic Construct
  VARIANT 3: note = Xaa = Asp or Leu
  VARIANT 5: note = Xaa = Phe or Tyr
  VARIANT 10: note = Xaa = Val or Tyr
ARXGXGYFDX

SEQ ID NO: 9 - protein, 12 aa, synthetic construct
  REGION 1..12: note = Synthetic Construct
  VARIANT 3: note = Xaa = Leu or Arg
  VARIANT 4: note = Xaa = Ile or Pro
  VARIANT 5: note = Xaa = Ala or Tyr
  VARIANT 6: note = Xaa = Ser or Thr
  VARIANT 8: note = Xaa = Thr or Tyr
  VARIANT 9: note = Xaa = Ala or Tyr
ARXXXXAXXFDY

SEQ ID NO: 10 - protein, 17 aa, synthetic construct
  REGION 1..17: note = Synthetic Construct
  VARIANT 4: note = Xaa = Ile or Val
  VARIANT 5: note = Xaa = Ala or His
  VARIANT 6: note = Xaa = Pro or Ser
  VARIANT 13: note = Xaa = Asp or Tyr
  VARIANT 17: note = Xaa = Phe or Val
ARDXXXGSSGYYXGFDX

SEQ ID NO: 11 - protein, 11 aa, synthetic construct
  REGION 1..11: note = Synthetic Construct
  VARIANT 5: note = Xaa = Gly or Ser
  VARIANT 7: note = Xaa = Gly or Ser
  VARIANT 6: note = Xaa = Ile or Val
  VARIANT 9: note = Xaa = Ser or Tyr
  VARIANT 11: note = Xaa = Ala or Asn
RASQXXXSXLX

SEQ ID NO: 12 - protein, 12 aa, synthetic construct
  REGION 1..12: note = Synthetic Construct
  VARIANT 5: note = Xaa = Ser or Thr
  VARIANT 7: note = Xaa = Phe, Arg, or Ser
  VARIANT 8: note = Xaa = Gly or Ser
  VARIANT 10: note = Xaa = Phe or Tyr
RASQXVXXRXLA

SEQ ID NO: 13 - protein, 15 aa, synthetic construct
  REGION 1..15: note = Synthetic Construct
  VARIANT 4: note = Xaa = Glu or Gln
  VARIANT 9: note = Xaa = Asp, Phe, His, or Tyr
  VARIANT 11: note = Xaa = Phe, Ile, or Lys
  VARIANT 15: note = Xaa = Ala, Asp, or His
RASXSVDFXGXSFLX

SEQ ID NO: 14 - skipped

SEQ ID NO: 15 - protein, 11 aa, synthetic construct
  REGION 1..11: note = Synthetic Construct
  VARIANT 3: note = Xaa = Glu, Gln, or Val
  VARIANT 4: note = Xaa = His or Gln
  VARIANT 5: note = Xaa = Ala, Gly, His, Arg, or Ser
  VARIANT 6: note = Xaa = Asp, Leu, Ser, or Tyr
  VARIANT 7: note = Xaa = Glu, Gly, Pro, Gln, or Ser
  VARIANT 8: note = Xaa = Leu, Thr, Val, or Trp
  VARIANT 10: note = Xaa = Phe, Leu, Pro, Trp, or Tyr
YCXXXXXXPXT

SEQ ID NO: 16 - protein, 12 aa, synthetic construct
  REGION 1..12: note = Synthetic Construct
  VARIANT 5: note = Xaa = Ser or Tyr
  VARIANT 6: note = Xaa = Asp or Tyr
  VARIANT 7: note = Xaa = Gln or Tyr
YCQQXXXWPPWT

SEQ ID NO: 17 - protein, 13 aa, synthetic construct
  REGION 1..13: note = Synthetic Construct
  VARIANT 4: note = Xaa = His or Gln
  VARIANT 6: note = Xaa = Thr or Val
  VARIANT 11: note = Xaa = Glu or Val
YCQXYXSSPPXYT

SEQ ID NO: 18 - protein, 11 aa, synthetic construct
  REGION 1..11: note = Synthetic Construct
FTFSDYAIHWV

SEQ ID NO: 19 - protein, 12 aa, synthetic construct
  REGION 1..12: note = Synthetic Construct
YSITSGYYWAWI

SEQ ID NO: 20 - protein, 11 aa, synthetic construct
  REGION 1..11: note = Synthetic Construct
FTFSDYGIHWV

SEQ ID NO: 21 - protein, 12 aa, synthetic construct
  REGION 1..12: note = Synthetic Construct
YSISSGYHWDWI

SEQ ID NO: 22 - protein, 11 aa, synthetic construct
  REGION 1..11: note = Synthetic Construct
FTFSDYWIHWV

SEQ ID NO: 23 - protein, 12 aa, synthetic construct
  REGION 1..12: note = Synthetic Construct
YSISSGYHWSWI

SEQ ID NO: 24 - protein, 13 aa, synthetic construct
  REGION 1..13: note = Synthetic Construct
FSLSTGGVAVSWI

SEQ ID NO: 25 - protein, 13 aa, synthetic construct
  REGION 1..13: note = Synthetic Construct
FSLSTGGVAVGWI

SEQ ID NO: 26 - protein, 11 aa, synthetic construct
  REGION 1..11: note = Synthetic Construct
FTFSGYAIHWV

SEQ ID NO: 27 - protein, 11 aa, synthetic construct
  REGION 1..11: note = Synthetic Construct
YTFSGYGIHWV

SEQ ID NO: 28 - protein, 11 aa, synthetic construct
  REGION 1..11: note = Synthetic Construct
YTFSGYAIHWV

SEQ ID NO: 29 - protein, 12 aa, synthetic construct
  REGION 1..12: note = Synthetic Construct
YSITSGHYWSWI

SEQ ID NO: 30 - protein, 21 aa, synthetic construct
  REGION 1..21: note = Synthetic Construct
IGIISPSSGSTNYAQKFQGRV

SEQ ID NO: 31 - protein, 21 aa, synthetic construct
  REGION 1..21: note = Synthetic Construct
VSSISGSGSTTYYADSVKGRF

SEQ ID NO: 32 - protein, 20 aa, synthetic construct
  REGION 1..20: note = Synthetic Construct
IGEIYHSGSTYYSPSLKSRV

SEQ ID NO: 33 - protein, 21 aa, synthetic construct
  REGION 1..21: note = Synthetic Construct
VSGISGYGGSTYYADSVKGRF

SEQ ID NO: 34 - protein, 21 aa, synthetic construct
  REGION 1..21: note = Synthetic Construct
IGWISPSGGGTKYAQKFQGRV

SEQ ID NO: 35 - protein, 20 aa, synthetic construct
  REGION 1..20: note = Synthetic Construct
LARIDWDDDKYYSTSLKSRL

SEQ ID NO: 36 - protein, 21 aa, synthetic construct
  REGION 1..21: note = Synthetic Construct
VSAISGYGSTTYYADSVKGRF

SEQ ID NO: 37 - protein, 21 aa, synthetic construct
  REGION 1..21: note = Synthetic Construct
IGIISPSGGGTKYAQKFQGRV

SEQ ID NO: 38 - protein, 21 aa, synthetic construct
  REGION 1..21: note = Synthetic Construct
IGIISPSGGSTKYAQKFQGRV

SEQ ID NO: 39 - protein, 20 aa, synthetic construct
  REGION 1..20: note = Synthetic Construct
IGDISHSGSTYYSQSLKSRV

SEQ ID NO: 40 - protein, 17 aa, synthetic construct
  REGION 1..17: note = Synthetic Construct
ARDIHSGSSGYYYGFDV

SEQ ID NO: 41 - protein, 10 aa, synthetic construct
  REGION 1..10: note = Synthetic Construct
ARDGFGYFDY

SEQ ID NO: 42 - protein, 17 aa, synthetic construct
  REGION 1..17: note = Synthetic Construct
ARDVAPGSSGYYDGFDF

SEQ ID NO: 43 - protein, 13 aa, synthetic construct
  REGION 1..13: note = Synthetic Construct
ARHSYYGSGNFDY

SEQ ID NO: 44 - protein, 9 aa, synthetic construct
  REGION 1..9: note = Synthetic Construct
ARGAYEFDY

SEQ ID NO: 45 - protein, 9 aa, synthetic construct
  REGION 1..9: note = Synthetic Construct
ARSYVYFDY

SEQ ID NO: 46 - protein, 12 aa, synthetic construct
  REGION 1..12: note = Synthetic Construct
ARRIATATYFDY

SEQ ID NO: 47 - protein, 12 aa, synthetic construct
  REGION 1..12: note = Synthetic Construct
ARLPYSAYAFDY

SEQ ID NO: 48 - protein, 7 aa, synthetic construct
  REGION 1..7: note = Synthetic Construct
ARHPFAY

SEQ ID NO: 49 - protein, 9 aa, synthetic construct
  REGION 1..9: note = Synthetic Construct
ARRIDAFDI

SEQ ID NO: 50 - protein, 8 aa, synthetic construct
  REGION 1..8: note = Synthetic Construct
ARLYDVAY

SEQ ID NO: 51 - protein, 10 aa, synthetic construct
  REGION 1..10: note = Synthetic Construct
ARLGYGYFDV

SEQ ID NO: 52 - protein, 11 aa, synthetic construct
  REGION 1..11: note = Synthetic Construct
ARGSRTGYFDY

SEQ ID NO: 53 - protein, 15 aa, synthetic construct
  REGION 1..15: note = Synthetic Construct
RASESVDFFGISFLA

SEQ ID NO: 54 - protein, 10 aa, synthetic construct
  REGION 1..10: note = Synthetic Construct
SASSSVSYVY

SEQ ID NO: 55 - protein, 11 aa, synthetic construct
  REGION 1..11: note = Synthetic Construct
RASQGIGSSLA

SEQ ID NO: 56 - protein, 15 aa, synthetic construct
  REGION 1..15: note = Synthetic Construct
RASESVDFFGKSFLH

SEQ ID NO: 57 - protein, 12 aa, synthetic construct
  REGION 1..12: note = Synthetic Construct
RASQSVSSRFLA

SEQ ID NO: 58 - protein, 12 aa, synthetic construct
  REGION 1..12: note = Synthetic Construct
RASQSVRGRFLA

SEQ ID NO: 59 - protein, 12 aa, synthetic construct
  REGION 1..12: note = Synthetic Construct
RASQTVFSRYLA

SEQ ID NO: 60 - protein, 11 aa, synthetic construct
  REGION 1..11: note = Synthetic Construct
RASQGVSSYLA

SEQ ID NO: 61 - protein, 15 aa, synthetic construct
  REGION 1..15: note = Synthetic Construct
RASQSVDFYGISFLD

SEQ ID NO: 62 - protein, 15 aa, synthetic construct
  REGION 1..15: note = Synthetic Construct
RASQSVDFDGFSFLH

SEQ ID NO: 63 - protein, 15 aa, synthetic construct
  REGION 1..15: note = Synthetic Construct
RASQSVDFHGKSFLH

SEQ ID NO: 64 - protein, 15 aa, synthetic construct
  REGION 1..15: note = Synthetic Construct
RASQSVDFYGISFLH

SEQ ID NO: 65 - protein, 11 aa, synthetic construct
  REGION 1..11: note = Synthetic Construct
RASQSISSYLN

SEQ ID NO: 66 - protein, 9 aa, synthetic construct
  REGION 1..9: note = Synthetic Construct
DASNRATGI

SEQ ID NO: 67 - protein, 9 aa, synthetic construct
  REGION 1..9: note = Synthetic Construct
DASSLESGV

SEQ ID NO: 68 - protein, 9 aa, synthetic construct
  REGION 1..9: note = Synthetic Construct
DASNLETGV

SEQ ID NO: 69 - protein, 9 aa, synthetic construct
  REGION 1..9: note = Synthetic Construct
AASTLQSGV

SEQ ID NO: 70 - protein, 13 aa, synthetic construct
  REGION 1..13: note = Synthetic Construct
YCQHYTSSPPVYT

SEQ ID NO: 71 - protein, 11 aa, synthetic construct
  REGION 1..11: note = Synthetic Construct
YCVQGLQTPWT

SEQ ID NO: 72 - protein, 12 aa, synthetic construct
  REGION 1..12: note = Synthetic Construct
YCQQYDQWPPWT

SEQ ID NO: 73 - protein, 11 aa, synthetic construct
  REGION 1..11: note = Synthetic Construct
YCQQSYSWPPT

SEQ ID NO: 74 - protein, 11 aa, synthetic construct
  REGION 1..11: note = Synthetic Construct
YCQQSYPTPLT

SEQ ID NO: 75 - protein, 11 aa, synthetic construct
  REGION 1..11: note = Synthetic Construct
YCQQSSSWPPT

SEQ ID NO: 76 - protein, 12 aa, synthetic construct
  REGION 1..12: note = Synthetic Construct
YCQQSYYWPPWT

SEQ ID NO: 77 - protein, 11 aa, synthetic construct
  REGION 1..11: note = Synthetic Construct
YCQHHYGTPLT

SEQ ID NO: 78 - protein, 13 aa, synthetic construct
  REGION 1..13: note = Synthetic Construct
YCQQYVSSPPEYT

SEQ ID NO: 79 - protein, 11 aa, synthetic construct
  REGION 1..11: note = Synthetic Construct
YCQQRDSWPYT

SEQ ID NO: 80 - protein, 11 aa, synthetic construct
  REGION 1..11: note = Synthetic Construct
YCEQSLEVPFT

SEQ ID NO: 81 - protein, 11 aa, synthetic construct
  REGION 1..11: note = Synthetic Construct
YCVQALQLPLT

SEQ ID NO: 82 - protein, 124 aa, synthetic construct
  REGION 1..124: note = Synthetic Construct
EVQLVESGGGLVQPGGSLRLSCAASGFTFSDYAIHWVRQAPGKGLEWIGIISPSSGSTNYAQKFQGRVTISRDNSKNTLYLQLNSLRAEDTAVYYCARDIHSGSSGYYYGFDVWGQGTLVTVSS

SEQ ID NO: 83 - protein, 118 aa, synthetic construct
  REGION 1..118: note = Synthetic Construct
EVQLVESGGGLVQPGGSLRLSCAASGYSITSGYYWAWIRQAPGKGLEWVSSISGSGSTTYYADSVKGRFTISRDNSKNTLYLQLNSLRAEDTAVYYCARDGFGYFDYWGQGTLVTVSS

SEQ ID NO: 84 - protein, 123 aa, synthetic construct
  REGION 1..123: note = Synthetic Construct
EVQLVESGGGLVQPGGSLRLSCAASGFTFSDYGIHWVRQAPGKGLEWIGEIYHSGSTYYSPSLKSRVTISRDNSKNTLYLQLNSLRAEDTAVYYCARDVAPGSSGYYDGFDFWGQGTLVTVSS

SEQ ID NO: 85 - protein, 121 aa, synthetic construct
  REGION 1..121: note = Synthetic Construct
EVQLVESGGGLVQPGGSLRLSCAASGYSISSGYHWDWIRQAPGKGLEWVSGISGYGGSTYYADSVKGRFTISRDNSKNTLYLQLNSLRAEDTAVYYCARHSYYGSGNFDYWGQGTLVTVSS

SEQ ID NO: 86 - protein, 116 aa, synthetic construct
  REGION 1..116: note = Synthetic Construct
EVQLVESGGGLVQPGGSLRLSCAASGFTFSDYWIHWVRQAPGKGLEWIGWISPSGGGTKYAQKFQGRVTISRDNSKNTLYLQLNSLRAEDTAVYYCARGAYEFDYWGQGTLVTVSS

SEQ ID NO: 87 - protein, 116 aa, synthetic construct
  REGION 1..116: note = Synthetic Construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSS

SEQ ID NO: 88 - protein, 120 aa, synthetic construct
  REGION 1..120: note = Synthetic Construct
EVQLVESGGGLVQPGGSLRLSCAASGFSLSTGGVAVSWIRQAPGKGLEWIGEIYHSGSTYYSPSLKSRVTISRDNSKNTLYLQLNSLRAEDTAVYYCARRIATATYFDYWGQGTLVTVSS

SEQ ID NO: 89 - protein, 121 aa, synthetic construct
  REGION 1..121: note = Synthetic Construct
EVQLVESGGGLVQPGGSLRLSCAASGFSLSTGGVAVGWIRQAPGKGLEWVSAISGYGSTTYYADSVKGRFTISRDNSKNTLYLQLNSLRAEDTAVYYCARLPYSAYAFDYWGQGTLVTVSS

SEQ ID NO: 90 - protein, 114 aa, synthetic construct
  REGION 1..114: note = Synthetic Construct
EVQLVESGGGLVQPGGSLRLSCAASGFTFSGYAIHWVRQAPGKGLEWIGIISPSGGGTKYAQKFQGRVTISRDNSKNTLYLQLNSLRAEDTAVYYCARHPFAYWGQGTLVTVSS

SEQ ID NO: 91 - protein, 115 aa, synthetic construct
  REGION 1..115: note = Synthetic Construct
EVQLVESGGGLVQPGGSLRLSCAASGYTFSGYGIHWVRQAPGKGLEWIGEIYHSGSTYYSPSLKSRVTISRDNSKNTLYLQLNSLRAEDTAVYYCARRIDAFDIWGQGTLVTVSS

SEQ ID NO: 92 - protein, 115 aa, synthetic construct
  REGION 1..115: note = Synthetic Construct
EVQLVESGGGLVQPGGSLRLSCAASGYTFSGYAIHWVRQAPGKGLEWIGIISPSGGGTKYAQKFQGRVTISRDNSKNTLYLQLNSLRAEDTAVYYCARLYDVAYWGQGTLVTVSS

SEQ ID NO: 93 - protein, 117 aa, synthetic construct
  REGION 1..117: note = Synthetic Construct
EVQLVESGGGLVQPGGSLRLSCAASGFTFSDYAIHWVRQAPGKGLEWIGIISPSGGSTKYAQKFQGRVTISRDNSKNTLYLQLNSLRAEDTAVYYCARLGYGYFDVWGQGTLVTVSS

SEQ ID NO: 94 - protein, 118 aa, synthetic construct
  REGION 1..118: note = Synthetic Construct
EVQLVESGGGLVQPGGSLRLSCAASGYSITSGHYWSWIRQAPGKGLEWIGDISHSGSTYYSQSLKSRVTISRDNSKNTLYLQLNSLRAEDTAVYYCARGSRTGYFDYWGQGTLVTVSS

SEQ ID NO: 95 - protein, 114 aa, synthetic construct
  REGION 1..114: note = Synthetic Construct
DIQLTQSPSSLSASVGDRVTITCRASESVDFFGISFLAWYQQKPGKAPKLLIYDASNRATGIPSRFSGSGSGTDFTLTISSLQPEDFATYYCQHYTSSPPVYTFGQGTKVEIKR

SEQ ID NO: 96 - protein, 107 aa, synthetic construct
  REGION 1..107: note = Synthetic Construct
DIQLTQSPSSLSASVGDRVTITCSASSSVSYVYWYQQKPGKAPKLLIYDASSLESGVPSRFSGSGSGTDFTLTISSLQPEDFATYYCVQGLQTPWTFGQGTKVEIKR

SEQ ID NO: 97 - protein, 109 aa, synthetic construct
  REGION 1..109: note = Synthetic Construct
DIQLTQSPSSLSASVGDRVTITCRASQGIGSSLAWYQQKPGKAPKLLIYDASNRATGIPSRFSGSGSGTDFTLTISSLQPEDFATYYCQQYDQWPPWTFGQGTKVEIKR

SEQ ID NO: 98 - protein, 112 aa, synthetic construct
  REGION 1..112: note = Synthetic Construct
DIQLTQSPSSLSASVGDRVTITCRASESVDFFGKSFLHWYQQKPGKAPKLLIYDASNLETGVPSRFSGSGSGTDFTLTISSLQPEDFATYYCQQSYSWPPTFGQGTKVEIKR

SEQ ID NO: 99 - protein, 109 aa, synthetic construct
  REGION 1..109: note = Synthetic Construct
DIQLTQSPSSLSASVGDRVTITCRASQSVSSRFLAWYQQKPGKAPKLLIYDASNRATGIPSRFSGSGSGTDFTLTISSLQPEDFATYYCQQSYPTPLTFGQGTKVEIKR

SEQ ID NO: 100 - protein, 109 aa, synthetic construct
  REGION 1..109: note = Synthetic Construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKR

SEQ ID NO: 101 - protein, 110 aa, synthetic construct
  REGION 1..110: note = Synthetic Construct
DIQLTQSPSSLSASVGDRVTITCRASQTVFSRYLAWYQQKPGKAPKLLIYDASNRATGIPSRFSGSGSGTDFTLTISSLQPEDFATYYCQQSYYWPPWTFGQGTKVEIKR

SEQ ID NO: 102 - protein, 108 aa, synthetic construct
  REGION 1..108: note = Synthetic Construct
DIQLTQSPSSLSASVGDRVTITCRASQGVSSYLAWYQQKPGKAPKLLIYAASTLQSGVPSRFSGSGSGTDFTLTISSLQPEDFATYYCQHHYGTPLTFGQGTKVEIKR

SEQ ID NO: 103 - protein, 114 aa, synthetic construct
  REGION 1..114: note = Synthetic Construct
DIQLTQSPSSLSASVGDRVTITCRASQSVDFYGISFLDWYQQKPGKAPKLLIYDASNRATGIPSRFSGSGSGTDFTLTISSLQPEDFATYYCQQYVSSPPEYTFGQGTKVEIKR

SEQ ID NO: 104 - protein, 112 aa, synthetic construct
  REGION 1..112: note = Synthetic Construct
DIQLTQSPSSLSASVGDRVTITCRASQSVDFDGFSFLHWYQQKPGKAPKLLIYDASSLESGVPSRFSGSGSGTDFTLTISSLQPEDFATYYCQQRDSWPYTFGQGTKVEIKR

SEQ ID NO: 105 - protein, 112 aa, synthetic construct
  REGION 1..112: note = Synthetic Construct
DIQLTQSPSSLSASVGDRVTITCRASQSVDFHGKSFLHWYQQKPGKAPKLLIYDASSLESGVPSRFSGSGSGTDFTLTISSLQPEDFATYYCEQSLEVPFTFGQGTKVEIKR

SEQ ID NO: 106 - protein, 112 aa, synthetic construct
  REGION 1..112: note = Synthetic Construct
DIQLTQSPSSLSASVGDRVTITCRASQSVDFYGISFLHWYQQKPGKAPKLLIYDASSLESGVPSRFSGSGSGTDFTLTISSLQPEDFATYYCVQALQLPLTFGQGTKVEIKR

SEQ ID NO: 107 - protein, 108 aa, synthetic construct
  REGION 1..108: note = Synthetic Construct
DIQLTQSPSSLSASVGDRVTITCRASQSISSYLNWYQQKPGKAPKLLIYDASNLETGVPSRFSGSGSGTDFTLTISSLQPEDFATYYCQHHYGTPLTFGQGTKVEIKR

SEQ ID NO: 108 - protein, 120 aa, synthetic construct
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPE

SEQ ID NO: 109 - protein, 5 aa, synthetic construct
DYEMH

SEQ ID NO: 110 - protein, 17 aa, synthetic construct
VIESETGGTAYNQKFKG

SEQ ID NO: 111 - protein, 16 aa, synthetic construct
EGITTVATTYYWYFDV

SEQ ID NO: 112 - protein, 16 aa, synthetic construct
RSSQSIVHSNGNTYLE

SEQ ID NO: 113 - protein, 7 aa, synthetic construct
KVSNRFS

SEQ ID NO: 114 - protein, 9 aa, synthetic construct
FQGSHVPLT

SEQ ID NO: 115 - protein, 125 aa, synthetic construct [sequence omitted]

SEQ ID NO: 116 - protein, 112 aa, synthetic construct [sequence omitted]

SEQ ID NO: 117-124 - skipped (8 entries)

SEQ ID NO: 125 - protein, 445 aa, synthetic construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 126 - protein, 446 aa, synthetic construct
EVQLVESGGGLVQPGGSLRLSCAASGYSISSGYHWSWIRQAPGKGLEWLARIDWDDDKYYSTSLKSRLTISRDNSKNTLYLQLNSLRAEDTAVYYCARSYVYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 215 aa, synthetic construct
DIQLTQSPSSLSASVGDRVTITCRASQSVRGRFLAWYQQKPGKAPKLLIYDASNRATGIPSRFSGSGSGTDFTLTISSLQPEDFATYYCQQSSSWPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 128 - protein, 446 aa, synthetic construct
EVQLVESGGGLVQPGGSLRLSCAASGFTFSDYAIHWVRQAPGKGLEWIGIISPSGGSTKYAQKFQGRVTISRDNSKNTLYLQLNSLRAEDTAVYYCARLGYGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 129 - protein, 447 aa, synthetic construct
EVQLVESGGGLVQPGGSLRLSCAASGFTFSDYAIHWVRQAPGKGLEWIGIISPSGGSTKYAQKFQGRVTISRDNSKNTLYLQLNSLRAEDTAVYYCARLGYGYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 218 aa, synthetic construct
DIQLTQSPSSLSASVGDRVTITCRASQSVDFYGISFLHWYQQKPGKAPKLLIYDASSLESGVPSRFSGSGSGTDFTLTISSLQPEDFATYYCVQALQLPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 447 aa, synthetic construct
EVQLVESGGGLVQPGGSLRLSCAASGYSITSGYYWAWIRQAPGKGLEWVSSISGSGSTTYYADSVKGRFTISRDNSKNTLYLQLNSLRAEDTAVYYCARDGFGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 132 - protein, 448 aa, synthetic construct
EVQLVESGGGLVQPGGSLRLSCAASGYSITSGYYWAWIRQAPGKGLEWVSSISGSGSTTYYADSVKGRFTISRDNSKNTLYLQLNSLRAEDTAVYYCARDGFGYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 213 aa, synthetic construct
DIQLTQSPSSLSASVGDRVTITCSASSSVSYVYWYQQKPGKAPKLLIYDASSLESGVPSRFSGSGSGTDFTLTISSLQPEDFATYYCVQGLQTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 443 aa, synthetic construct
EVQLVESGGGLVQPGGSLRLSCAASGFTFSGYAIHWVRQAPGKGLEWIGIISPSGGGTKYAQKFQGRVTISRDNSKNTLYLQLNSLRAEDTAVYYCARHPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 135 - protein, 444 aa, synthetic construct
EVQLVESGGGLVQPGGSLRLSCAASGFTFSGYAIHWVRQAPGKGLEWIGIISPSGGGTKYAQKFQGRVTISRDNSKNTLYLQLNSLRAEDTAVYYCARHPFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 220 aa, synthetic construct
DIQLTQSPSSLSASVGDRVTITCRASQSVDFYGISFLDWYQQKPGKAPKLLIYDASNRATGIPSRFSGSGSGTDFTLTISSLQPEDFATYYCQQYVSSPPEYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 444 aa, synthetic construct
EVQLVESGGGLVQPGGSLRLSCAASGYTFSGYAIHWVRQAPGKGLEWIGIISPSGGGTKYAQKFQGRVTISRDNSKNTLYLQLNSLRAEDTAVYYCARLYDVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 138 - protein, 445 aa, synthetic construct
EVQLVESGGGLVQPGGSLRLSCAASGYTFSGYAIHWVRQAPGKGLEWIGIISPSGGGTKYAQKFQGRVTISRDNSKNTLYLQLNSLRAEDTAVYYCARLYDVAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 218 aa, synthetic construct
DIQLTQSPSSLSASVGDRVTITCRASQSVDFHGKSFLHWYQQKPGKAPKLLIYDASSLESGVPSRFSGSGSGTDFTLTISSLQPEDFATYYCEQSLEVPFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC